(19) United States
(12) Patent Application Publication
(10) Pub. No.: US 2021/0179586 A1
(43) Pub. Date: Jun. 17, 2021

(54) SOLID STATE FORMS OF BRANAPLAM AND THEIR PREPARATION

(71) Applicant: TEVA PHARMACEUTICALS INTERNATIONAL GMBH, Jona (CH)

(72) Inventors: Luna Ben-Sahel Katsav, Petah Tikva (IL); Doron Rudik, Modi'in (IL); Firas Masri, Nazareth (IL); Motti Erlich, Petah Tikva (IL); Polina Lapido, Rishon LeZion (IL); Ofir Shaul, Hod Hasharon (IL); Sigalit Patael, Petah Tikva (IL)

(21) Appl. No.: 17/269,040

(22) PCT Filed: Sep. 5, 2019
(86) PCT No.: PCT/US19/49669

§ 371 Date: Feb. 17, 2021

Related U.S. Application Data
(60) Provisional application No. 62/728,390, filed on Sep. 7, 2018.
(60) Provisional application No. 62/756,618, filed on Nov. 7, 2018.
(60) Provisional application No. 62/770,242, filed on Nov. 21, 2018.
(60) Provisional application No. 62/782,422, filed on Dec. 20, 2018.

(51) Int. Cl. C07D 401/14

(52) U.S. Cl. CPC C07D 401/14; C07B 2200/13

(57) ABSTRACT

The present disclosure relates to Branaplam solid state forms, Branaplam salts and solid states thereof, processes for preparation thereof, pharmaceutical compositions and methods of use thereof.

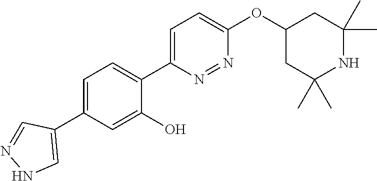 [SMILES: CC1(C)CC(Oc2ccc(-c3ccc(-c4cn[nH]c4)cc3O)nn2)CC(C)(C)N1]

### TECHNICAL FIELD

The present disclosure encompasses crystalline polymorphs of Branaplam, processes for preparation thereof, and pharmaceutical compositions thereof.

### BACKGROUND

Branaplam, chemical name (6E)-3-(1H-pyrazol-4-yl)-6-[3-(2,2,6,6-tetramethylpiperidin-4-yl)oxy-1H-pyridazin-6-ylidene]cyclohexa-2,4-dien-1-one, has the following chemical structure:

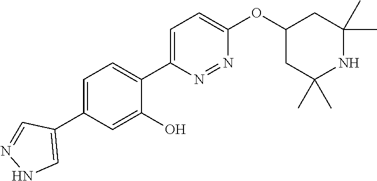 [SMILES: CC1(C)CC(Oc2ccc(-c3ccc(-c4cn[nH]c4)cc3O)nn2)CC(C)(C)N1]

Branaplam, also known as LMI070, is an orally available drug which aims to correct the splicing of SMN2, the “back-up” gene, thereby increasing the amount of SMN protein made. Branaplam is being developed by Novartis for the treatment of spinal muscular atrophy (SMA).

Branaplam compound and process for its preparation are described in U.S. Pat. Nos. 8,729,263 and 9,545,404 and in CN patent publication 103,965,169. However, the reported yield for Branaplam hydrochloride in U.S. Pat. Nos. 8,729,263 and 9,545,404 is very poor (9%, Example 17-13).

Polymorphism, the occurrence of different crystalline forms, is a property of some molecules and molecular complexes. A single molecule, like Branaplam, may give rise to a variety of polymorphs having distinct crystal structures and physical properties like melting point, thermal behaviors (e.g., measured by thermogravimetric analysis—“TGA”, or differential scanning calorimetry—“DSC”), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, improving the dissolution profile, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient to provide an improved product.

Discovering new solid state forms and solvates of a pharmaceutical product may yield materials having desirable processing properties, such as ease of handling, ease of processing, storage stability, and ease of purification, or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, e.g., a different crystal habit, higher crystallinity, or polymorphic stability, which may offer better processing or handling characteristics, improved dissolution profile, or improved shelf-life (chemical/physical stability).

For at least these reasons, there is a need for additional solid state forms (including solvated forms) of Branaplam.

### SUMMARY OF THE DISCLOSURE

The present disclosure provides crystalline polymorphs of Branaplam and salts thereof including hydrochloride and sulfate, processes for preparation thereof, and pharmaceutical compositions thereof. These crystalline polymorphs can be used to prepare other forms of Branaplam and salts thereof, including hydrochloride and sulfate.

In particular, the present disclosure relates to solid state forms of Branaplam designated as Form B1, Form B3 and Form B5 (defined herein), solid state forms of Branaplam hydrochloride salt designated as Forms C1, C2, C3, C6, C8, C9, C10 and C11 (defined herein), and to solid state form of Branaplam sulfate salt Form S1 (defined herein). The present disclosure also provides uses of any one or combination of the above described solid state forms of Branaplam and salts thereof, including hydrochloride and sulfate, for preparing other solid state forms of Branaplam and salts thereof.

The present disclosure further provides processes for preparing Branaplam and salts thereof, including hydrochloride and sulfate and solid state forms thereof.

In another embodiment, the present disclosure provides any one of the above described crystalline polymorphs of Branaplam and salts thereof, including hydrochloride and sulfate, for use in medicine, in embodiments for the treatment of spinal muscular atrophy (SMA).

The present disclosure also encompasses the uses of any one of the above described crystalline polymorphs of Branaplam and salts thereof, including hydrochloride and sulfate of the present disclosure, for the preparation of pharmaceutical compositions and/or formulations.

In another aspect, the present disclosure provides pharmaceutical compositions including crystalline polymorphs of Branaplam and salts thereof, including hydrochloride and sulfate, according to the present disclosure.

In yet another embodiment, the present disclosure encompasses pharmaceutical formulations including any one of the above described crystalline polymorphs of Branaplam and salts thereof, including hydrochloride and sulfate and/or combinations thereof, and at least one pharmaceutically acceptable excipient.

The present disclosure encompasses processes for preparing the above mentioned pharmaceutical formulations of Branaplam and salts thereof, including hydrochloride and sulfate, which includes any one of the above described crystalline polymorphs and at least one pharmaceutically acceptable excipient.

The crystalline polymorphs defined herein and/or combinations thereof as well as the pharmaceutical compositions or formulations of the crystalline polymorphs of Branaplam and salts thereof, including hydrochloride and sulfate, may be used as medicaments, in embodiments for the treatment of spinal muscular atrophy (SMA).

The present disclosure also provides methods of treating spinal muscular atrophy (SMA), by administering a therapeutically effective amount of any one of the crystalline polymorphs of Branaplam and salts thereof, including hydrochloride and sulfate of the present disclosure, or at least one of the above pharmaceutical compositions or formulations, to a subject suffering from spinal muscular atrophy (SMA) or otherwise in need of the treatment.

The present disclosure also provides the uses of any one of the solid state forms of Branaplam and salts thereof, including hydrochloride and sulfate of the present disclosure, or at least one of the above pharmaceutical compositions or formulations, for the manufacture of medicaments for treating spinal muscular atrophy (SMA).

### BRIEF DESCRIPTION OF THE DRAWINGS

FIG. 1 shows a characteristic X-ray powder diffraction pattern (XRPD) of Branaplam Form B1.

FIG. 2 shows a characteristic X-ray powder diffraction pattern (XRPD) of Branaplam Form B3.

FIG. 3 shows a characteristic X-ray powder diffraction pattern (XRPD) of Branaplam Form B5.

FIG. 4 shows a characteristic X-ray powder diffraction pattern (XRPD) of Branaplam hydrochloride salt Form C1.

FIG. 5 shows a characteristic X-ray powder diffraction pattern (XRPD) of Branaplam hydrochloride salt Form C3 (peak at 28.5° 2 theta is attribute to addition of Si).

FIG. 6 shows a characteristic X-ray powder diffraction pattern (XRPD) of Branaplam hydrochloride salt Form C6,

FIG. 7 shows a characteristic X-ray powder diffraction pattern (XRPD) of Branaplam hydrochloride salt Form C2.

FIG. 8 shows characteristic X-ray powder diffraction pattern (XRPD) of Branaplam hydrochloride salt Form C8.

FIG. 9 shows a characteristic X-ray powder diffraction pattern (XRPD) of Branaplam hydrochloride salt Form C9.

FIG. 10 shows a characteristic X-ray powder diffraction pattern (XRPD) of amorphous Branaplam hydrochloride salt.

FIG. 11 shows a characteristic X-ray powder diffraction pattern (XRPD) of Branaplam hydrochloride salt form C10.

FIG. 12 shows a characteristic X-ray powder diffraction pattern (XRPD) of Branaplam hydrochloride salt form C11.

FIG. 13 shows a characteristic X-ray powder diffraction pattern (XRPD) of Branaplam Sulfate salt form S1.

FIG. 14 shows a solid state ¹³C NMR spectrum of form C1 of Branaplam hydrochloride at the range of 200-0 ppm.

FIG. 15 shows a solid state ¹³C NMR spectrum of form C1 of Branaplam hydrochloride at the range of 200-100 ppm.

FIG. 16 shows a solid state ¹³C NMR spectrum of form C1 of Branaplam hydrochloride at the range of 100-0 ppm.

FIG. 17 shows a solid state ¹³C NMR spectrum of form C3 of Branaplam hydrochloride at the range of 200-0 ppm.

FIG. 18 shows a solid state ¹³C NMR spectrum of form C3 of Branaplam hydrochloride at the range of 200-100 ppm.

FIG. 19 shows a solid state ¹³C NMR spectrum of form C3 of Branaplam hydrochloride at the range of 100-0 ppm.

FIG. 20 shows a solid state ¹³C NMR spectrum of form C10 of Branaplam hydrochloride at the range of 200-0 ppm.

FIG. 21 shows a solid state ¹³C NMR spectrum of form C10 of Branaplam hydrochloride at the range of 200-100 ppm.

FIG. 22 shows a solid state ¹³C NMR spectrum of form C10 of Branaplam hydrochloride at the range of 100-0 ppm.

### DETAILED DESCRIPTION OF THE DISCLOSURE

The present disclosure encompasses crystalline polymorphs of Branaplam and salts thereof, processes for preparation thereof, and pharmaceutical compositions including at least one of, or a combination of, these solid state forms. The disclosure also relates to the conversion of Branaplam and its solid state forms to other solid state forms of Branaplam and salts thereof.

The Branaplam, Branaplam salts, and solid state forms thereof according to the present disclosure may have advantageous properties selected from at least one of the following: chemical or polymorphic purity, flowability, solubility, dissolution rate, bioavailability, morphology or crystal habit, stability—such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility, and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Branaplam referred to herein as being characterized by graphical data “as depicted in” or “as substantially depicted in” a Figure will thus be understood to include any crystal forms of Branaplam characterized with the graphical data having such small variations, as are well known to the skilled person, in comparison with the Figure.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0.5% or less or about 0% of any other forms of the subject compound as measured, for example, by XRPD. Thus, solid state form of Branaplam described herein as substantially free of any other solid state forms would be understood to contain greater than about 80% (w/w), greater than about 90% (w/w), greater than about 95% (w/w), greater than about 98% (w/w), greater than about 99% (w/w), greater than about 99.5%, or about 100% of the subject crystalline polymorph of Branaplam. In some embodiments of the disclosure, the described crystalline polymorph of Branaplam may contain from about 0.5% to about 20% (w/w), from about 5% to about 20% (w/w), or from about 5% to about 10% (w/w) of one or more other solid state forms of Branaplam.

As used herein, unless stated otherwise, reference to % values are to wt %. This is based on the assumption that the solvent % in the various forms is measured in wt %.

As used herein, and unless stated otherwise, the term “anhydrous” in relation to crystalline forms of Branaplam, relates to a crystalline form of Branaplam which does not include any crystalline water (or other solvents) in a defined, stoichiometric amount within the crystal. Moreover, an “anhydrous” form would typically not contain more than 1% (w/w), of either water or organic solvents as measured for example by TGA (Thermal Gravimetric Analysis).

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein the term non-hygroscopic in relation to crystalline Branaplam and salts thereof refers to less than 0.2% (w/w) absorption of water by the crystalline Branaplam, when exposed to 80% relative humidity (“RH”) for 24 hours at room temperature (“RT”), as determined, for example, by TGA.

As used herein the term “stable” in relation to crystalline Branaplam and salts thereof refers to less than 20%, less than 10%, less than 5%, less than 2%, less than 1%, or less than 0.5% conversion of crystalline Branaplam and salts thereof to any other solid state form of Branaplam when kept in storage for long term stability e.g. 60% RH/RT or 75% RH/40° C. for at least 1 month. Alternatively, crystalline Branaplam and salts thereof are stable when exposed to high humidity e.g. 80-100% RH for 7 days, high temperature e.g. 100° C. for 30 minutes, high mass pressure e.g. 3 Ton for 1 minute or upon strong grinding.

As used herein, the term “isolated” in reference to crystalline polymorph of Branaplam of the present disclosure corresponds to a crystalline polymorph of Branaplam that is physically separated from the reaction mixture in which it is formed.

As used herein, unless stated otherwise, the XRPD measurements are taken using copper Kα radiation wavelength 1.54060 Å. XRPD peaks reported herein are measured using CuK α radiation, 1.54060 Å, at a temperature of 25±3° C.

As used herein, unless stated otherwise, ¹³C solid state NMR was measured at 400 MHz at room temperature at a spin rate of 11 kHz.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, typically about 16 hours.

As used herein, the term “reduced pressure” refers to a pressure that is less than atmospheric pressure. For example, reduced pressure is about 10 mbar to about 50 mbar.

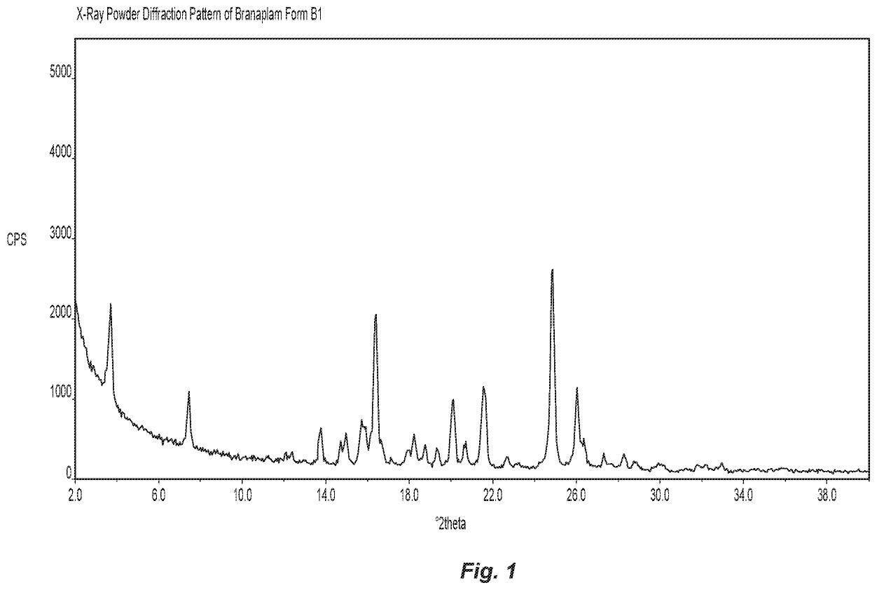

The present disclosure includes a crystalline polymorph of Branaplam, designated Form B1. The crystalline Form B1 of Branaplam may be characterized by data selected from one or more of the following: an X-ray powder diffraction pattern substantially as depicted in FIG. 1; an X-ray powder diffraction pattern having peaks at 3.7, 7.5, 16.4, 24.8 and 26.0 degrees 2-theta±0.2 degrees 2-theta; and combinations of these data.

Crystalline Form B1 of Branaplam may be further characterized by an X-ray powder diffraction pattern having peaks at 3.7, 7.5, 16.4, 24.8 and 26.0 degrees 2-theta±0.2 degrees 2-theta, and also having any one, two, three, four or five additional peaks selected from 13.8, 15.0, 18.2, 20.1 and 20.7 degrees 2-theta±0.2 degrees 2-theta.

Crystalline Form B1 of Branaplam may alternatively be characterized by an XRPD pattern having peaks at 3.7, 7.5, 13.8, 15.0, 16.4, 18.2, 20.1, 20.7, 24.8 and 26.0 degrees 2-theta±0.2 degrees 2-theta.

Crystalline Form B1 of Branaplam may be characterized by each of the above characteristics alone/or by all possible combinations, e.g., an XRPD pattern having peaks at 3.7, 7.5, 16.4, 24.8 and 26.0 degrees 2-theta±0.2 degrees 2-theta; an XRPD pattern as depicted in FIG. 1, and combinations thereof.

In another embodiment of the present disclosure, Branaplam Form B1 is polymorphically pure.

In an embodiment of the present disclosure, crystalline Form B1 of Branaplam is isolated.

Branaplam Form B1 may be characterized as hydrate form. In certain embodiments, Form B1 may contain from about 2% to about 4% of water by weight, in embodiments from about 3% to about 4%, and in other embodiments about 3.5% of water by weight as determined, for example, by TGA or by any other suitable techniques.

As described above, depending on which other solid state it is compared with, Form B1 of Branaplam according to the present disclosure may have advantageous properties as described above. For example, Branaplam Form B1 is stable and non-hygroscopic.

Branaplam Form B1 may be prepared by a process including crystallising an aqueous suspension including Branaplam, optionally isolating the Branaplam Form B1, and optionally drying.

The above Branaplam Form B1 can be prepared by a process including: suspending Branaplam hydrochloride (in embodiments Branaplam hydrochloride Form C1) in water and adding basic solution to the suspension. In embodiments, the aqueous suspension including Branaplam is prepared by basifying a suspension of Branaplam hydrochloride, in embodiments Form C1 as defined herein.

Typically, about 10 to about 40 vol of water, in embodiments double distilled water, in embodiments about 15 to about 30 vol of water, in embodiments about 15 to about 25 vol and in other embodiments about 20 to about 25 vol of water, is used for preparing Branaplam hydrochloride suspension.

The suspension is kept at about 70 to about 90° C., in embodiments at about 75 to about 85° C. and in other embodiments at about 80° C., in embodiments for a time period of about 5 to about 60 minutes, in embodiments for about 5 to about 30 minutes and in other embodiments for about 5 minutes, optionally with stirring.

Typically, the basifying is carried out using an aqueous solution of an alkali metal hydroxide, in embodiments selected from: potassium hydroxide, sodium hydroxide, or lithium hydroxide, in embodiments wherein the alkali metal hydroxide is sodium hydroxide. In embodiments, the aqueous solution of alkali metal hydroxide is added to the above suspension.

About 0.8 to about 1.5 molar equivalents of sodium hydroxide, in embodiments about 0.8 to about 1.2 molar equivalents, in embodiments about 0.8 to about 1.0 molar equivalents, and in other embodiments about 0.9 molar equivalents, of sodium hydroxide is added to the suspension until a pH of about 9 is reached.

Before cooling to room temperature the suspension is kept at the same temperature for additional about 5 to about 30 minutes, in embodiments for about 5 to about 15 and in other embodiments for about 5 minutes, optionally with stirring.

In embodiments, isolation of Branaplam Form B1 may be done, for example, by cooling the suspension to room temperature, filtering the resulting suspension and optionally drying. Drying may be done by nitrogen or air or under vacuum. In embodiments, drying is performed at a temperature of about 20 to about 50° C., about 40 to about 50° C., in embodiments about 45° C. When the drying is under vacuum, in embodiments a reduced pressure of: about 1 to about 200 mbar, about 1 to about 100 mbar, about 1 to about 50 mbar, about 1 to about 20 mbar or in other embodiments about 10 mbar, may be used.

In another embodiment the above Branaplam Form B1 can be also prepared by a process including a solvent/antisolvent crystallization. In embodiments, Branaplam Form B1 is prepared by crystallization from a mixture of 2,2,2-trifluoroethanol and water. In some embodiments, the process includes:

a) combining a solution of Branaplam in 2,2,2-trifluoroethanol, optionally with heating, with water;
b) optionally isolating the Branaplam Form B1, in embodiments by filtration; and
c) optionally drying.

In embodiments, the solution in stage a) includes Branaplam and 2,2,2-Trifluoroethanol, wherein the 2,2,2-Trifluoroethanol is present at an amount of about 15 to about 35 vol, in embodiments about 20 to about 30 vol, and in yet other embodiments, about 25 vol of 2,2,2-Trifluoroethanol, by heating to a temperature of about 50 to about 78° C., in embodiments about 70 to about 78° C., and in other embodiments to about 75° C., optionally with stirring, optionally using mechanically filtration.

Step (a) may include simultaneous addition of water to the solution of Branaplam in 2,2,2-trifluoroethanol, in embodiments step (a) includes simultaneous dropwise addition of the solution of Branaplam in 2,2,2-trifluoroethanol and water, in embodiments at the same rate, into a reaction vessel. The mixture may be stirred during the addition. In embodiments, the volume ratio of 2,2,2-trifluoroethanol to water is: about 1:1 to about 1:2, about 1:1 to about 1:1.5 and in some embodiments about 1:1.

In embodiments, the Branaplam solution and water are simultaneously added drop-wise into a vessel which is maintained at room temperature. The addition may be carried out over a period of about 5 to about 30 minutes, about 5 to about 20 minutes, and in some embodiments within about 10 minutes, preferably with stirring.

Following the addition of the Branaplam solution and water, the mixture may be further maintained for about 1 to about 24 hours, about 10 to about 24 hours, or about 18 hours, preferably at room temperature, and preferably with stirring.

In any embodiment of the above process, step (b) may include isolation of Branaplam Form B1. The isolation may be done by filtering the suspension formed in step (a). Following the isolation, the product may be washed, and optionally dried. Drying may be done by nitrogen or air or under vacuum. Drying may be performed at a temperature of about 20 to about 50° C., about 40 to about 50° C., and in some embodiments about 45° C. When the drying is carried out under vacuum, a reduced pressure of: about 1 or about 200 mbar, about 1 to about 100 mbar, about 1 to about 50 mbar, and particularly about 5 to about 40 mbar or more particularly, about 20 mbar, is used.

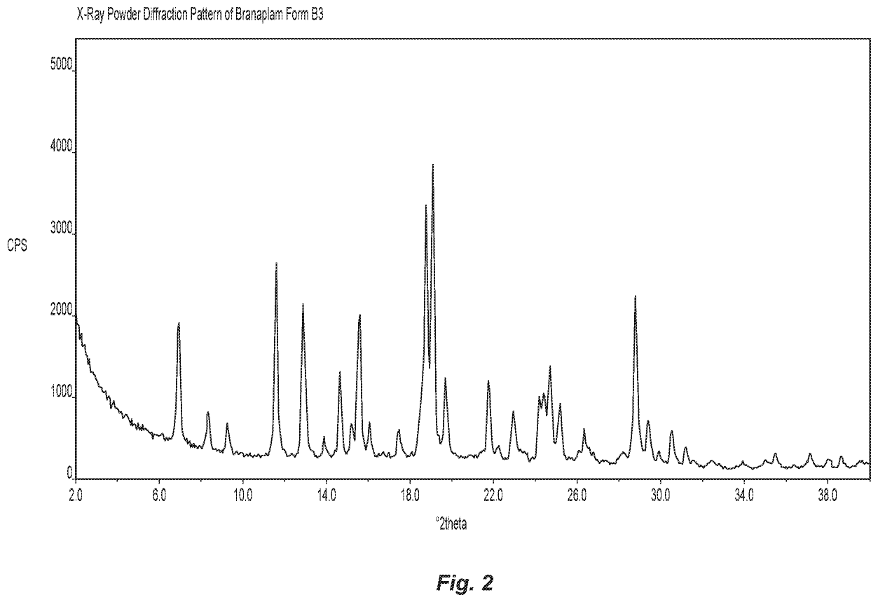

The present disclosure further includes a crystalline polymorph of Branaplam, designated Form B3. The crystalline Form B3 of Branaplam may be characterized by data selected from one or more of the following: an X-ray powder diffraction pattern substantially as depicted in FIG. 2; an X-ray powder diffraction pattern having peaks at 11.5, 12.9, 15.5, 19.0 and 28.8 degrees 2-theta±0.2 degrees 2-theta; and combinations of these data.

Crystalline Form B3 of Branaplam may be further characterized by an X-ray powder diffraction pattern having peaks at 11.5, 12.9, 15.5, 19.0 and 28.8 degrees 2-theta±0.2 degrees 2-theta, and also having any one, two, three, four or five additional peaks selected from 9.3, 14.6, 17.5, 19.8 and 21.7 degrees 2-theta±0.2 degrees 2-theta. In another embodiment Branaplam Form B3 can be characterized as anhydrous form.

Crystalline Form B3 of Branaplam may alternatively be characterized by an XRPD pattern having peaks at 9.3, 11.5, 12.9, 14.6, 15.5, 17.5, 19.0, 19.8, 21.7 and 28.8 degrees 2-theta±0.2 degrees 2-theta.

Crystalline Form B3 of Branaplam may be characterized by each of the above characteristics alone/or by all possible combinations, e.g., an XRPD pattern having peaks at 11.5, 12.9, 15.5, 19.0 and 28.8 degrees 2-theta±0.2 degrees 2-theta; an XRPD pattern as depicted in FIG. 2, and combinations thereof.

In another embodiment of the present disclosure, Branaplam Form B3 is polymorphically pure.

In an embodiment of the present disclosure, crystalline Form B3 of Branaplam is isolated.

As described above, depending on which other solid state it is compared with, Form B3 of Branaplam according to the present disclosure may have advantageous properties as described above. For example, Branaplam Form B3 is stable and non-hygroscopic.

The above Branaplam Form B3 can be prepared by a process including crystallization of Branaplam in DMAc (N,N-dimethylacetamide).

The process can include preparing a mixture of Branaplam in N,N-dimethylacetamide, optionally heating the mixture, and optionally isolating Branaplam Form B3.

In embodiments, about 15 to about 30 vol, about 15 to about 25 vol, and particularly about 20 vol, of DMAc relative to Branaplam, is used.

The mixture of Branaplam in N,N-dimethylacetamide may be heated to a temperature of about 90 to about 110° C., in embodiments about 100 to about 110° C. and in some embodiments about 110° C.

The mixture may be maintained at the elevated temperature for a period of: about 10 to about 120 minutes, about 10 to about 60 minutes, and particularly about 30 minutes, optionally with stirring. In embodiments, the hot mixture is filtered in order to obtain a filtrate which is a clear solution.

The clear solution may be cooled prior to isolating the Branaplam Form B3. In embodiments, the hot solution is cooled to a temperature of about 25 to about −5° C., in embodiments about 25 to about 0° C., in other embodiments to 20 to about 25° C., in embodiments to room temperature, optionally with stirring. Typically, the cooled mixture is a suspension.

Prior to isolation of the Branaplam Form B3, the suspension is maintained for an additional about 1 to about 24 hours, in embodiments about 10 to about 20 hours, in embodiments about 18 hours, in some embodiments at room temperature, optionally with stirring.

In embodiments, isolation of Branaplam Form B3 may be done, for example, by filtering the resulting suspension and optionally drying. Drying may be done by nitrogen or air or under vacuum. In embodiments, drying is performed at a temperature of about 40 to about 80° C., about 40 to about 60° C., about 40 about 50° C., or in some embodiments about 45° C. When the drying is carried out under vacuum, such as a reduced pressure of: about 1 to about 200 mbar, about 1 to about 100 mbar, about 1 to about 50 mbar, and in some embodiments about 1 to about 20 mbar or in one embodiment, about 10 mbar, is used.

Alternatively, the present disclosure further includes a process for the preparation of Branaplam Form B3 including a solvent/antisolvent crystallization. In this embodiment, Branaplam Form B3 is prepared by crystallization from a mixture of dimethyl sulfoxide (DMSO) and methyl acetate. The process includes:

a) providing a solution of Branaplam in DMSO (Dimethyl Sulfoxide), and optionally heating;
b) combining the solution with methyl acetate;
c) cooling, and optionally isolating, in embodiments by filtration, and optionally drying.

Step (a) can include dissolving Branaplam in DMSO, optionally with heating. In embodiments, the DMSO is used in amount of about 15 to about 30 vol, about 15 to about 25 vol, or particularly about 20 vol of DMSO. In embodiments, the mixture is heated to a temperature of about 80 to about 120° C., about 100 to about 120° C., and in some embodiments about 120° C., optionally with stirring. The hot mixture may be filtered prior to step (b).

In embodiments step (b) includes adding methyl acetate to the solution of Branaplam in DMSO. In embodiments, about 10 to about 30 vol, about 10 to about 25 vol, about 10 to about 20 vol, and particularly about 10 vol of methyl acetate is added. The addition may be carried out in one portion, optionally with stirring.

After the addition of the methyl acetate, in embodiments the mixture is spontaneously cooled to room temperature (i.e., after the addition of the methyl acetate, the mixture is allowed to cool to room temperature). The mixture is maintained for an additional about 1 to about 24 hours, about 10 to about 24 hours, or about 16 hours, in embodiments under stirring, at room temperature.

The Branaplam Form B3 may be isolated, in embodiments by filtration. The product may be washed and optionally dried. Drying may be done by nitrogen or air or under vacuum. In embodiments, drying may be performed at a temperature of about 40 to about 60° C., about 40 to about 55° C., and particularly about 45° C. When the drying is under vacuum, such as a reduced pressure of: about 1 to about 200 mbar, about 1 to 100 mbar, about 1 to about 50 mbar, and particularly about 5 to about 40 mbar, or more particularly, about 20 mbar, is used.

Alternatively, Branaplam Form B3 may be prepared by heating Branaplam Form B1 to about 250° C. using a heating rate of about 10° C./min, in embodiments under nitrogen, optionally using a DSC instrument.

In an aspect, about 2 to about 10 mg Branaplam Form B1, in embodiments about 3 to about 10 mg and in some embodiments about 5 mg is heated under nitrogen. Optionally the heating is carried out under a nitrogen flow, in embodiments using about 50 ml/min nitrogen flow.

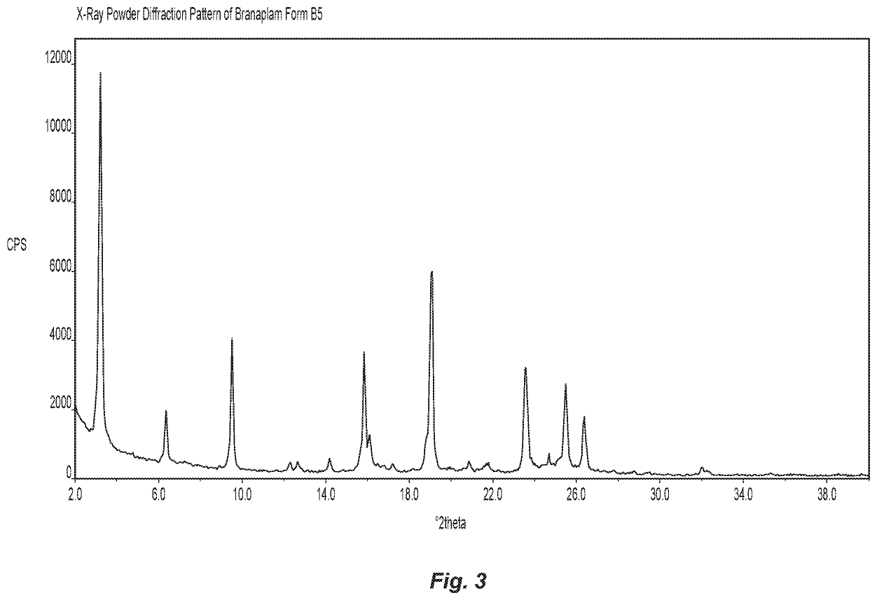

The present disclosure includes a crystalline polymorph of Branaplam, designated Form B5. The crystalline Form B5 of Branaplam may be characterized by data selected from one or more of the following: an X-ray powder diffraction pattern substantially as depicted in FIG. 3; an X-ray powder diffraction pattern having peaks at 6.4, 9.5, 15.9, 19.0 and 23.5 degrees 2-theta±0.2 degrees 2-theta; and combinations of these data.

Crystalline Form B5 of Branaplam may be further characterized by an X-ray powder diffraction pattern having peaks at 6.4, 9.5, 15.9, 19.0 and 23.5 degrees 2-theta±0.2 degrees 2-theta, and also having any one, two, three, four or five additional peaks selected from 3.2, 14.2, 20.9, 25.5 and 26.4 degrees 2-theta±0.2 degrees 2-theta. In another embodiment Branaplam Form B5 can be characterized as solvate, in embodiments a 2,2,2-Trifluoroethanol solvate.

Crystalline Form B5 of Branaplam may alternatively be characterized by an XRPD pattern having peaks at 3.2, 6.4, 9.5, 14.2, 15.9, 19.0, 20.9, 23.5, 25.5 and 26.4 degrees 2-theta±0.2 degrees 2-theta.

Crystalline Form B5 of Branaplam may be characterized by each of the above characteristics alone/or by all possible combinations, e.g., an XRPD pattern having peaks at 6.4, 9.5, 15.9, 19.0 and 23.5 degrees 2-theta±0.2 degrees 2-theta; an XRPD pattern as depicted in FIG. 3, and combinations thereof.

In another embodiment of the present disclosure, Branaplam Form B5 is polymorphically pure.

In an embodiment of the present disclosure, crystalline Form B5 of Branaplam is isolated.

The above Branaplam Form B5 can be prepared by a process including crystallization of Branaplam in 2,2,2-Trifluoroethanol, optionally isolating the Branaplam Form B5, and optionally drying.

In embodiments, about 70 to about 150 vol, about 90 to about 120 vol, about 90 to about 110 vol, and in some embodiments about 106 vol of 2,2,2-Trifluoroethanol is used to dissolve Branaplam. The solution is preferably maintained at room temperature, in embodiments at 20 to about 25° C., optionally with stirring.

In embodiments, the solution is kept under the above described conditions for about 5 to about 60 minutes, about 10 to about 30 minutes, and in embodiments about 10 minutes, optionally with stirring. The solution may be filtered.

In embodiments, the Branaplam Form B5 is crystallised by solvent removal. In embodiments, the solvent removal is carried out by slow evaporation, in embodiments at room temperature for about 3 to about 14 days, in embodiments about 3 to about 10 days, and in some embodiments about 5 days.

In embodiments, the resulting solid is dried. Drying may be done by nitrogen or air or under vacuum. In embodiments, drying is performed at a temperature of about 20 to about 60° C., about 40 to about 60° C., about 40 to about 55° C., or about 45° C. When the drying is under vacuum, in embodiments a reduced pressure of: about 1 to about 200 mbar, about 1 to about 100 mbar, about 1 to about 50 mbar, particularly about 1 to about 20 mbar and more particularly about 10 mbar, is used.

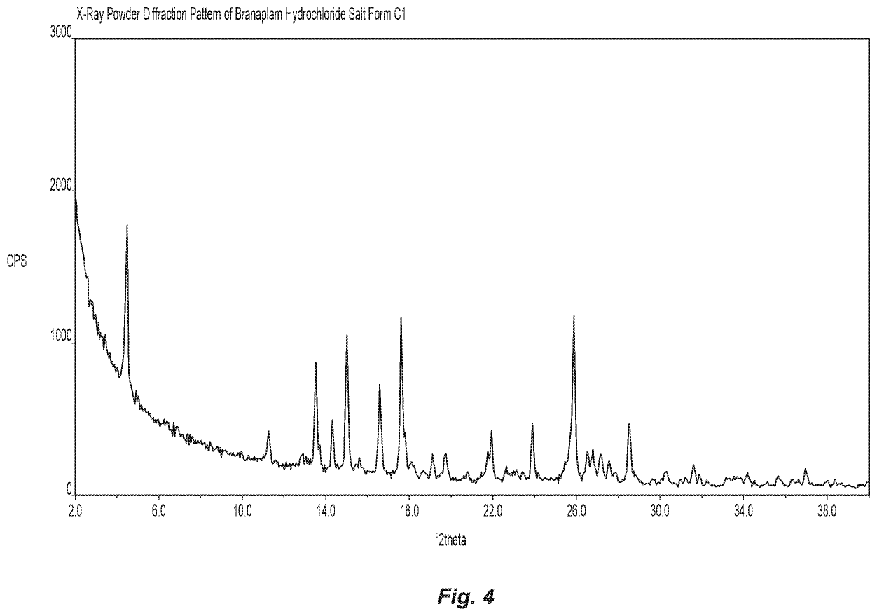

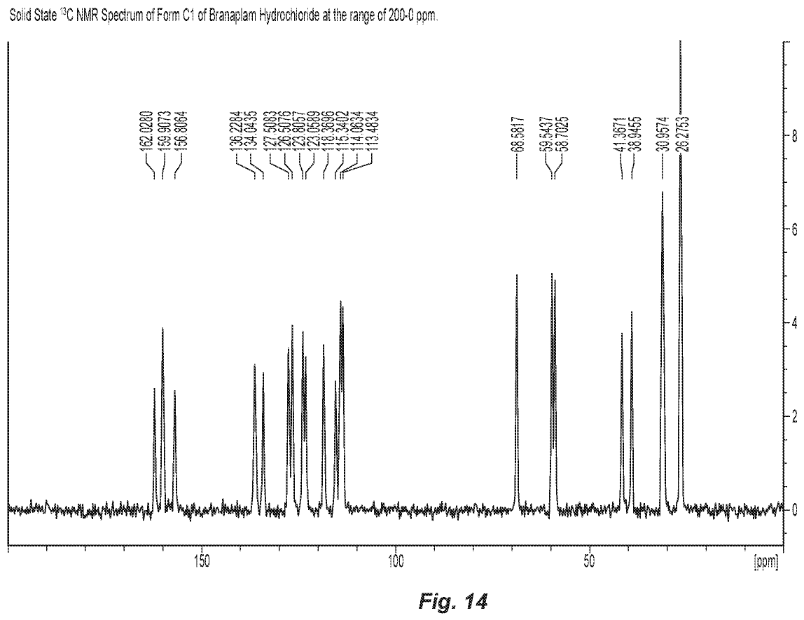

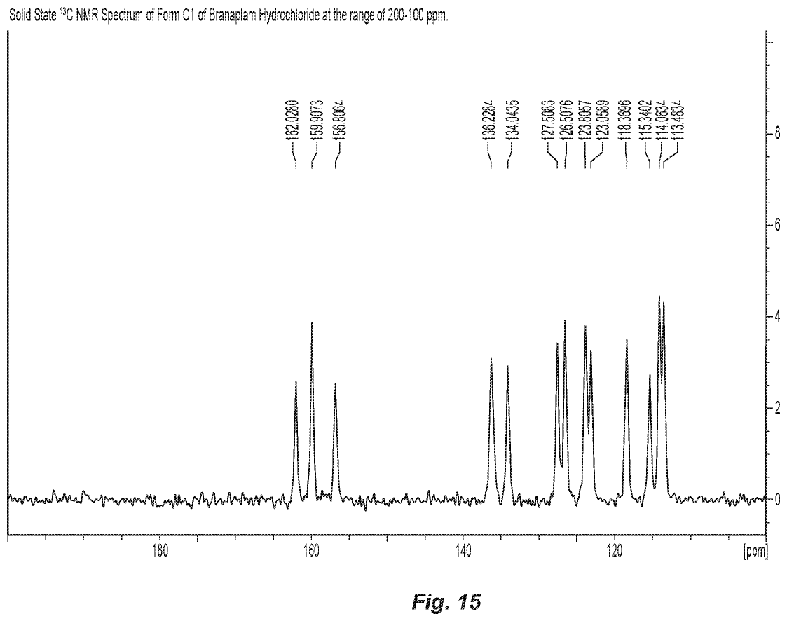

The present disclosure includes a crystalline polymorph of Branaplam hydrochloride, designated Form C1. The crystalline Form C1 of Branaplam hydrochloride may be characterized by data selected from one or more of the following: an X-ray powder diffraction pattern substantially as depicted in FIG. 4; an X-ray powder diffraction pattern having peaks at 4.5, 15.0, 16.6, 17.7 and 25.9 degrees 2-theta±0.2 degrees 2-theta; a solid state ¹³C-NMR spectrum having characteristic peaks at 160.0, 134.1, 126.5, 118.4 and 113.6 ppm±0.2 ppm; a solid state ¹³C-NMR spectrum having the following chemical shift absolute differences from a reference peak at 162.1 ppm±2 ppm of 2.1, 28.0, 35.6, 43.7 and 48.5 ppm±0.1 ppm; a solid state ¹³C-NMR spectrum as depicted in FIG. 14 or 15 or 16; and combinations of these data.

Crystalline Form C1 of Branaplam hydrochloride may be further characterized by an X-ray powder diffraction pattern having peaks at 4.5, 15.0, 16.6, 17.7 and 25.9 degrees 2-theta±0.2 degrees 2-theta, and also having any one, two, three, four or five additional peaks selected from 11.3, 13.5, 14.3, 24.0 and 28.5 degrees 2-theta±0.2 degrees 2-theta.

Crystalline Form C1 of Branaplam hydrochloride may alternatively be characterized by an XRPD pattern having peaks at 4.5, 11.3, 13.5, 14.3, 15.0, 16.6, 17.7, 24.0, 25.9 and 28.5 degrees 2-theta±0.2 degrees 2-theta.

Crystalline Form C1 of Branaplam hydrochloride may be characterized by each of the above characteristics alone/or by all possible combinations, e.g., an XRPD pattern having peaks at 4.5, 15.0, 16.6, 17.7 and 25.9 degrees 2-theta±0.2 degrees 2-theta; an XRPD pattern as depicted in FIG. 4, and combinations thereof.

In one embodiment Branaplam hydrochloride Form C1 can be characterized as hydrate, such as monohydrate. In certain embodiments, Branaplam hydrochloride Form C1 contains about 3-5% of water, in embodiments about 4.3% (theoretical water content for monohydrate is 4.0%).

As described above, depending on which other solid state it is compared with, Form C1 of Branaplam hydrochloride according to the present disclosure may have advantageous properties as described above. For example, Branaplam hydrochloride Form C1 is surprisingly stable and non-hygroscopic.

Form C1 of Branaplam hydrochloride can be prepared by a process including crystallising Branaplam hydrochloride in either ethanol or water.

In one embodiment, Form C1 of Branaplam hydrochloride can be prepared by a process including crystallising Branaplam hydrochloride from ethanol, optionally isolating the Branaplam hydrochloride Form C1, and optionally drying.

In embodiments, the process includes suspending Branaplam (such as Branaplam Forms B1 or B3, as defined herein or mixtures thereof) in ethanol, and adding a solution of HCl in ethanol.

In embodiments, the ethanol is used in an amount of about 8 to about 20 vol of ethanol, in some embodiment about 8 to about 15 vol of ethanol, in other embodiments about 8 to about 12 vol, and in an aspect about 10 vol of ethanol is used for preparing Branaplam suspension. The ethanol may be absolute ethanol.

In embodiments, the solution of HCl gas in ethanol is added in one portion to the suspension. Typically, about 1 to about 1.65 molar equivalents (in relation to Branaplam free base) of the HCl solution, in embodiments about 1.5 molar equivalents of the HCl solution is added to the suspension. Optionally, another portion of ethanol may be added, typically in an amount of about 2 to about 5 vol, and in some embodiments about 3 vol or about 2.7 vol.

In embodiments, the resulting suspension is maintained at room temperature, for additional 1 to about 20 hours, in embodiments for about 10 to about 20 hours, and in some embodiments for about 18 hours, optionally with stirring.

In embodiments, the suspension is cooled to about 0° C. to about 5° C., in embodiments to about 0° C. The suspension may be maintained at the temperature for about 15 to about 120 minutes, about 15 to about 60 minutes, and in some embodiments for 30 minutes, optionally with stirring.

In an embodiment, Branaplam hydrochloride Form C1 may be isolated, for example, by filtering the suspension, optionally washing with ethanol and optional drying. Drying may be done by nitrogen or air or under vacuum. In embodiments, drying is performed at a temperature of about 30 to about 80° C., about 40 to about 60° C., about 40 to about 50° C., about 45° C. When the drying is under vacuum, in embodiments a reduced pressure of: about 1 to about 200 mbar, about 1 to about 100 mbar, about 1 to about 50 mbar, particularly about 1 to about 20 mbar or more particularly about 10 mbar, is used.

The above Branaplam hydrochloride Form C1 may be prepared by crystallization of Branaplam hydrochloride (such as Branaplam Form C6 as defined herein) in water.

The process may include suspending Branaplam hydrochloride (such as Branaplam Form C6 as defined herein) in water, in embodiments at room temperature.

About 10 to about 30 vol of water, in embodiments about 15 to about 25 vol of water, and in other embodiments about 20 vol of water may be used for preparing Branaplam suspension. The water may be double distilled.

In embodiments, prior to isolation, the suspension is maintained at room temperature for about 1 to about 10 hours, in other embodiments about 5 to about 10 hours, and in other embodiments about 8 to about 9 hours, in some embodiments about 8.5 hours, optionally with stirring.

In embodiments, the Branaplam hydrochloride Form C1 is isolated, for example by filtration. The isolated product may be optionally dried. Drying may be done by nitrogen or air or under vacuum. In embodiments, drying is performed at a temperature of about 30 to about 80° C., about 40 to about 50° C., about 40 to about 50° C., or particularly about 45° C. When the drying is under vacuum, it may be at a reduced pressure of: about 1 to about 200 mbar, about 1 to about 100 mbar, about 1 to about 50 mbar, in embodiments about 1 to about 20 mbar or in some embodiments about 10 mbar, is used.

Alternatively, the preparation of Branaplam hydrochloride Form C1 may include crystallization of Branaplam hydrochloride in a mixture of DMSO and water. The process may include:

a) combining a solution of Branaplam hydrochloride in DMSO (dimethyl sulfoxide), optionally with heating, with water;
b) optionally cooling, and
c) optionally isolating the Branaplam hydrochloride Form C1.

Typically, about 5 to about 12 vol DMSO is used in stage a), in embodiments about 5 to about 10 vol, in some embodiments about 7 vol. The mixture may be heated to a temperature of about 80 to about 120° C. in embodiments about 110° C. The solution may be stirred, and filtered prior to combining with water.

In embodiments, water is added to the solution of Branaplam hydrochloride in DMSO. In embodiments, the volume ratio of DMSO to water is: about 1:1 to about 1:2, about 1:1 to about 1:1.8 and in some embodiments about 1:1.5 (v/v). In embodiments, the water is added dropwise to the solution.

In embodiments, the suspension obtained in stage a) is cooled, in some embodiments to room temperature. The solution may be maintained for about 5 to about 24 hours, about 8 to about 15 hours, and particularly about 11 hours, optionally under stirring. Isolation of Branaplam hydrochloride Form C1 may be done for example by filtering the suspension, optionally drying. Drying may be done by nitrogen or air or under vacuum. Drying may be performed at a temperature of about 40 to about 60° C., in some embodiments about 40 to about 50° C., and particularly about 45° C. When the drying is under vacuum, in embodiments a reduced pressure of: about 1 to about 200 mbar, about 1 to about 100 mbar, about 1 to about 50 mbar, and particularly about 5 to about 40 mbar, and more particularly about 20 mbar, is used.

In another embodiment of the present disclosure, Branaplam hydrochloride Form C1 is polymorphically pure.

In an embodiment of the present disclosure, crystalline Form C1 of Branaplam hydrochloride is isolated.

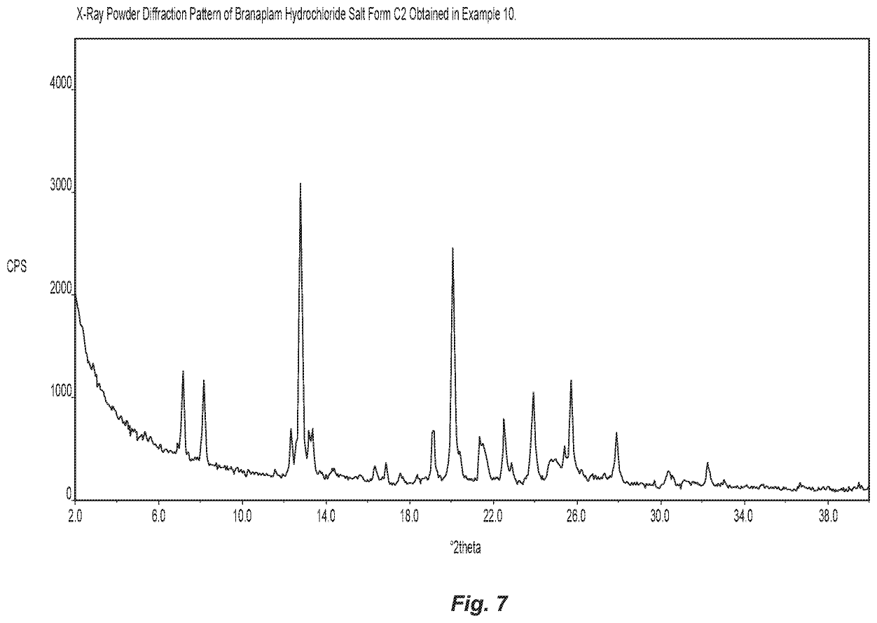

The present disclosure includes a crystalline polymorph of Branaplam hydrochloride, designated Form C2. The crystalline Form C2 of Branaplam hydrochloride may be characterized by data selected from one or more of the following: an X-ray powder diffraction pattern substantially as depicted in FIG. 7; an X-ray powder diffraction pattern having peaks at 7.2, 8.2, 12.8, 20.1 and 23.9 degrees 2-theta±0.2 degrees 2-theta; and combinations of these data.

Crystalline Form C2 of Branaplam hydrochloride may be further characterized by an X-ray powder diffraction pattern having peaks at 7.2, 8.2, 12.8, 20.1 and 23.9 degrees 2-theta±0.2 degrees 2-theta, and also having any one, two, three, four or five additional peaks selected from 19.1, 21.3, 22.5, 25.7 and 27.9 degrees 2-theta±0.2 degrees 2-theta.

Crystalline Form C2 of Branaplam hydrochloride may alternatively be characterized by an XRPD pattern having peaks at 7.2, 8.2, 12.8, 19.1, 20.1, 21.3, 22.5, 23.9, 25.7 and 27.9 degrees 2-theta±0.2 degrees 2-theta.

Crystalline Form C2 of Branaplam hydrochloride may be characterized by each of the above characteristics alone/or by all possible combinations, e.g., an XRPD pattern having peaks at 7.2, 8.2, 12.8, 20.1 and 23.9 degrees 2-theta±0.2 degrees 2-theta; an XRPD pattern as depicted in FIG. 7, and combinations thereof.

In another embodiment of the present disclosure, Branaplam hydrochloride Form C2 is polymorphically pure.

In an embodiment of the present disclosure, crystalline Form C2 of Branaplam hydrochloride is isolated.

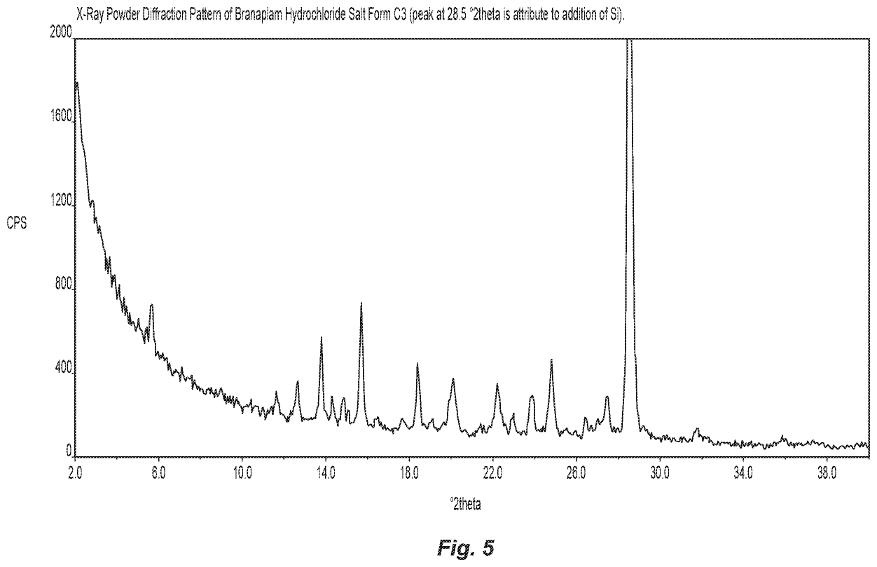

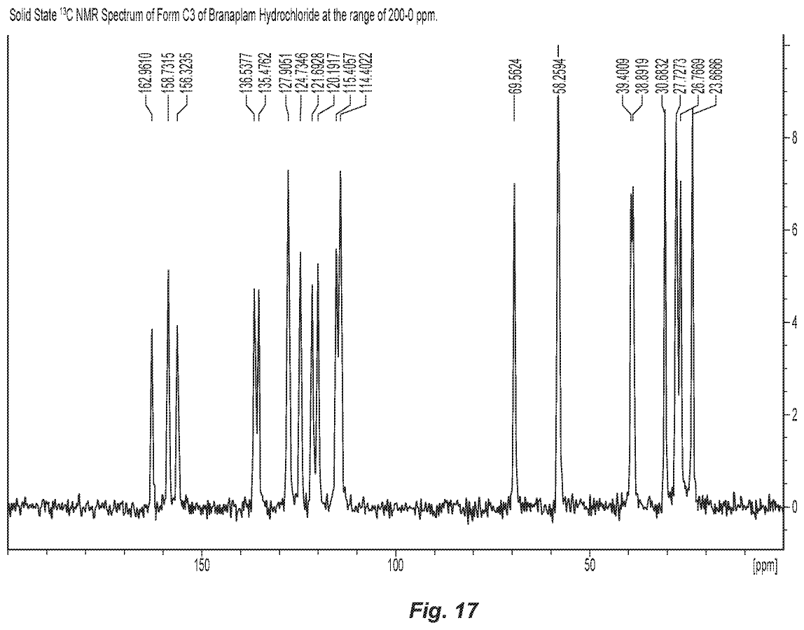

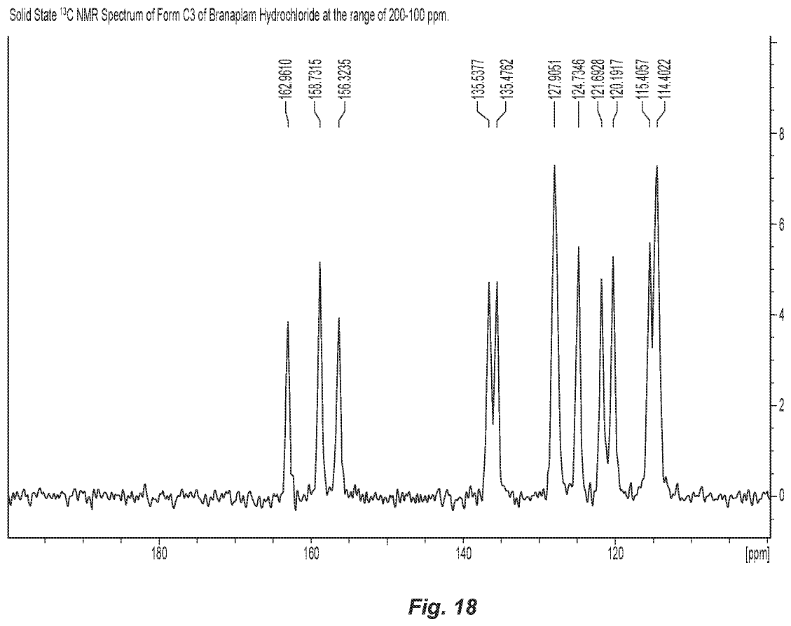

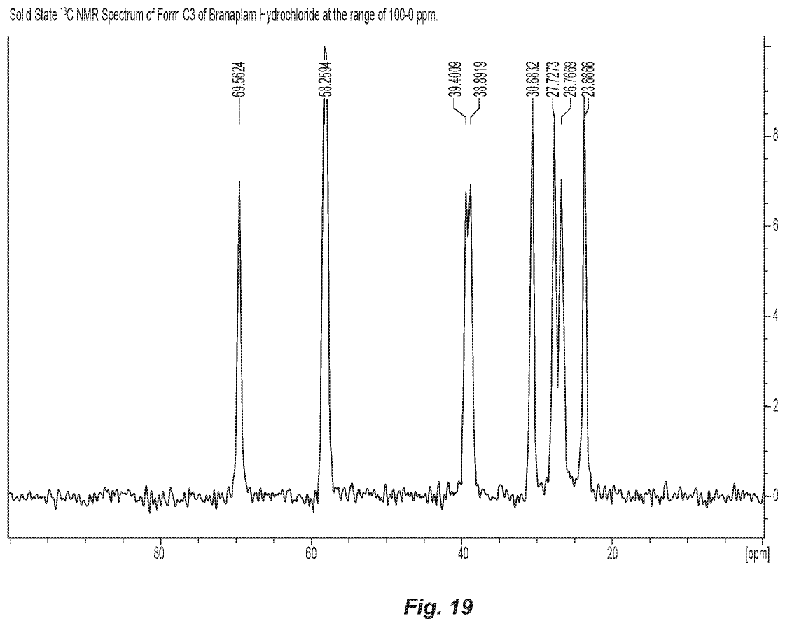

The present disclosure includes a crystalline polymorph of Branaplam hydrochloride, designated Form C3. The crystalline Form C3 of Branaplam hydrochloride may be characterized by data selected from one or more of the following: an X-ray powder diffraction pattern substantially as depicted in FIG. 5; an X-ray powder diffraction pattern having peaks at 5.6, 13.7, 15.6, 18.3 and 24.7 degrees 2-theta±0.2 degrees 2-theta; a solid state ¹³C-NMR spectrum having characteristic peaks at 156.4, 135.6, 124.8, 121.8 and 120.2 ppm±0.2 ppm; a solid state ¹³C-NMR spectrum having the following chemical shift absolute differences from a reference peak at 163.0 ppm±2 ppm of 6.6, 27.4, 38.2, 41.2 and 42.8 ppm±0.1 ppm; a solid state ¹³C-NMR spectrum as depicted in FIG. 17 or 18 or 19; and combinations of these data.

Crystalline Form C3 of Branaplam hydrochloride may be further characterized by an X-ray powder diffraction pattern having peaks at 5.6, 13.7, 15.6, 18.3 and 24.7 degrees 2-theta±0.2 degrees 2-theta, and also having any one, two, three, four or five additional peaks selected from 12.6, 14.7, 20.0, 22.1 and 23.8 degrees 2-theta±0.2 degrees 2-theta.

Crystalline Form C3 of Branaplam hydrochloride may alternatively be characterized by an XRPD pattern having peaks at 5.6, 12.6, 13.7, 14.7, 15.6, 18.3, 20.0, 22.1, 23.8 and 24.7 degrees 2-theta±0.2 degrees 2-theta.

In another embodiment Branaplam hydrochloride Form C3 can be characterized as anhydrous form.

Crystalline Form C3 of Branaplam hydrochloride may be characterized by each of the above characteristics alone/or by all possible combinations, e.g., an XRPD pattern having peaks at 5.6, 13.7, 15.6, 18.3 and 24.7 degrees 2-theta±0.2 degrees 2-theta; an XRPD pattern as depicted in FIG. 5, and combinations thereof.

In another embodiment of the present disclosure, Branaplam hydrochloride Form C3 is polymorphically pure.

In an embodiment of the present disclosure, crystalline Form C3 of Branaplam hydrochloride is isolated.

As described above, depending on which other solid state it is compared with, Form C3 of Branaplam hydrochloride according to the present disclosure may have advantageous properties as described above. For example, Branaplam hydrochloride Form C3 is stable upon heating and humidity.

The above Branaplam hydrochloride Form C3 can be prepared by a process including crystallising Branaplam hydrochloride (such as Form C1) in acetonitrile. In embodiments, the process includes suspending Branaplam hydrochloride (such as Branaplam Form C1 as defined herein) in acetonitrile.

Typically, about 200 to about 500 vol of acetonitrile, in embodiments about 300 to about 400 vol, and in some embodiments about 400 vol of acetonitrile, is used for preparing Branaplam hydrochloride suspension.

In embodiments, the suspension is maintained at room temperature for about 2 to about 30 minutes, in embodiments about 2 to about 10 minutes and in some embodiments for about 5 minutes, optionally with stirring.

In embodiments, the suspension is heated to about 40 to about 80° C., in embodiments about 60° C., in embodiments for about 2 to about 30 minutes, in some embodiments for about 2 to about 10 minutes and in some embodiments for about 5 minutes, optionally with stirring.

The Branaplam hydrochloride may be isolated and dried. In embodiments, isolation of Branaplam hydrochloride Form C3 may be done, for example, by filtering the resulting suspension. In embodiments, the product is dried, in embodiments by nitrogen or air or under vacuum. In embodiments, drying is performed at a temperature of about 30 to about 80° C., about 40 to about 60° C., about 40 to about 50° C., and particularly about 45° C. When the drying is under vacuum, a reduced pressure of: about 1 to about 200 mbar, about 1 about 100 mbar, about 1 to about 50 mbar, and particularly about 1 to about 20 mbar and more particularly about 10 mbar, is used.

Alternatively, the above Branaplam hydrochloride Form C3 can be prepared by a process including crystallising Branaplam hydrochloride (such as Branaplam hydrochloride Form C1) in ethyl acetate. In embodiments, the process includes suspending Branaplam hydrochloride (such as Branaplam Form C1 as defined herein) in ethyl acetate.

Typically, about 15 to about 45 vol of ethyl acetate, in embodiments about 20 to about 40 vol, and in some embodiments about 30 vol of ethyl acetate is used for preparing the Branaplam hydrochloride suspension.

In embodiments, the suspension is heated to about 50 to about 90° C., in embodiments, about 65 to about 78° C., and in some embodiments to about 75° C., for additional about 10 to about 60 hours, in embodiments for about 20 to about 30 hours and in some embodiments for about 24 hours, optionally with stirring.

In embodiments, the Branaplam is isolated and optionally dried. The isolation of Branaplam hydrochloride Form C3 may be done, for example, by filtering the resulting suspension. Drying may be done by nitrogen or air or under vacuum. In embodiments, drying is performed at a temperature of about 30 to about 80° C., about 40 to about 60° C., about 40 to about 50° C., and particularly about 45° C. When the drying is under vacuum, in embodiments a reduced pressure of: about 1 to about 200 mbar, about 1 to about 100 mbar, about 1 to about 50 mbar, particularly about 1 to about 20 mbar or more particularly about 10 mbar, is used.

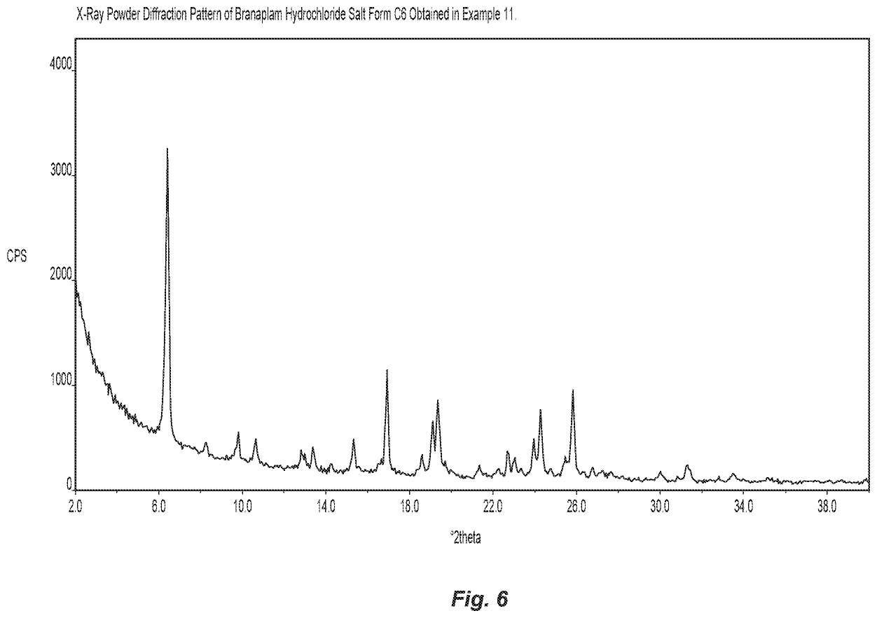

The present disclosure includes a crystalline polymorph of Branaplam hydrochloride, designated Form C6. The crystalline Form C6 of Branaplam hydrochloride may be characterized by data selected from one or more of the following: an X-ray powder diffraction pattern substantially as depicted in FIG. 6; an X-ray powder diffraction pattern having peaks at 6.4, 16.9, 19.4, 24.2 and 25.8 degrees 2-theta±0.2 degrees 2-theta; and combinations of these data.

Crystalline Form C6 of Branaplam hydrochloride may be further characterized by an X-ray powder diffraction pattern having peaks at 6.4, 16.9, 19.4, 24.2 and 25.8 degrees 2-theta±0.2 degrees 2-theta, and also having any one, two, three, four or five additional peaks selected from 8.2, 9.8, 10.6, 15.3, and 22.7 degrees 2-theta±0.2 degrees 2-theta.

Crystalline Form C6 of Branaplam hydrochloride may alternatively be characterized by an XRPD pattern having peaks at 6.4, 8.2, 9.8, 10.6, 15.3, 16.9, 19.4, 22.7, 24.2 and 25.8 degrees 2-theta±0.2 degrees 2-theta.

Crystalline Form C6 of Branaplam hydrochloride may be characterized by each of the above characteristics alone/or by all possible combinations, e.g., an XRPD pattern having peaks at 6.4, 16.9, 19.4, 24.2 and 25.8 degrees 2-theta±0.2 degrees 2-theta; an XRPD pattern as depicted in FIG. 6, and combinations thereof.

In another embodiment of the present disclosure, Branaplam hydrochloride Form C6 is polymorphically pure.

In an embodiment of the present disclosure, crystalline Form C6 of Branaplam hydrochloride is isolated.

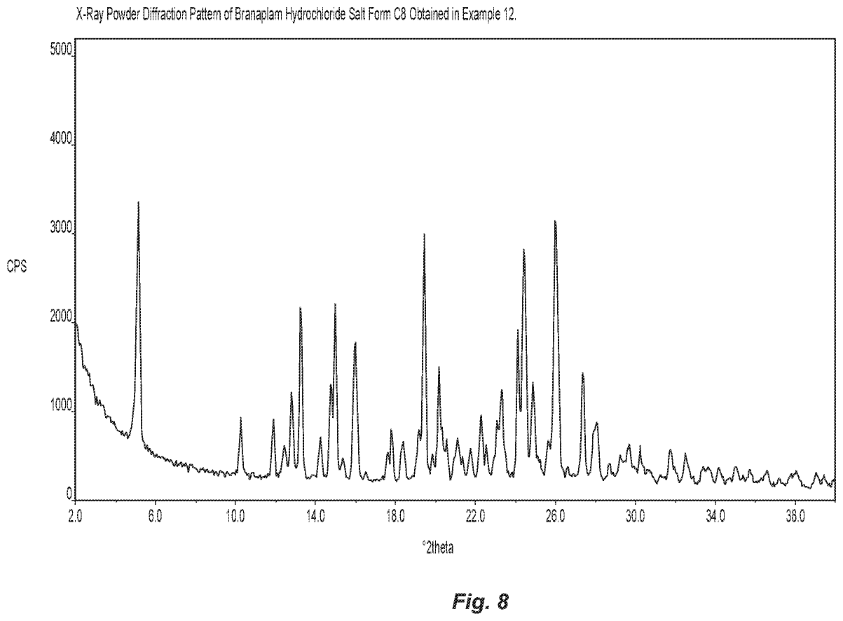

The present disclosure includes a crystalline polymorph of Branaplam hydrochloride, designated Form C8. The crystalline Form C8 of Branaplam hydrochloride may be characterized by data selected from one or more of the following: an X-ray powder diffraction pattern substantially as depicted in FIG. 8; an X-ray powder diffraction pattern having peaks at 5.0, 13.2, 15.9, 19.4 and 26.0 degrees 2-theta±0.2 degrees 2-theta; and combinations of these data.

Crystalline Form C8 of Branaplam hydrochloride may be further characterized by an X-ray powder diffraction pattern having peaks at 5.0, 13.2, 15.9, 19.4 and 26.0 degrees 2-theta±0.2 degrees 2-theta, and also having any one, two, three, four or five additional peaks selected from 10.2, 11.9, 15.0, 24.4 and 27.3 degrees 2-theta±0.2 degrees 2-theta.

Crystalline Form C8 of Branaplam hydrochloride may alternatively be characterized by an XRPD pattern having peaks at 5.0, 10.2, 11.9, 13.2, 15.0, 15.9, 19.4, 24.4, 26.0 and 27.3 degrees 2-theta±0.2 degrees 2-theta.

Crystalline Form C8 of Branaplam hydrochloride may be characterized by each of the above characteristics alone/or by all possible combinations, e.g., an XRPD pattern having peaks at 5.0, 13.2, 15.9, 19.4 and 26.0 degrees 2-theta±0.2 degrees 2-theta; an XRPD pattern as depicted in FIG. 8, and combinations thereof.

In another embodiment of the present disclosure, Branaplam hydrochloride Form C8 is polymorphically pure.

In an embodiment of the present disclosure, crystalline Form C8 of Branaplam hydrochloride is isolated.

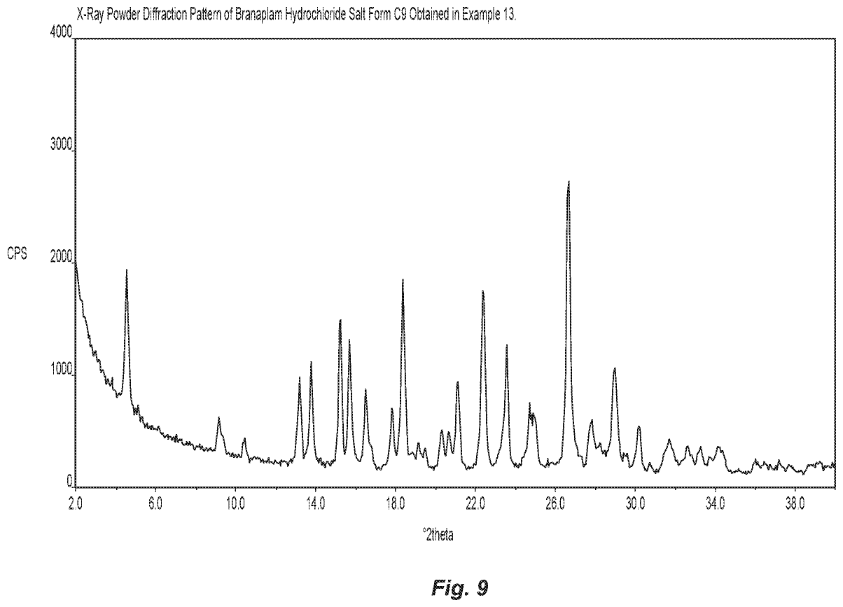

The present disclosure includes a crystalline polymorph of Branaplam hydrochloride, designated Form C9. The crystalline Form C9 of Branaplam hydrochloride may be characterized by data selected from one or more of the following: an X-ray powder diffraction pattern substantially as depicted in FIG. 9; an X-ray powder diffraction pattern having peaks at 4.6, 13.2, 13.8, 18.5 and 23.6 degrees 2-theta±0.2 degrees 2-theta; and combinations of these data.

Crystalline Form C9 of Branaplam hydrochloride may be further characterized by an X-ray powder diffraction pattern having peaks at 4.6, 13.2, 13.8, 18.5 and 23.6 degrees 2-theta±0.2 degrees 2-theta, and also having any one, two, three, four or five additional peaks selected from 9.1, 15.3, 15.7, 22.4 and 26.6 degrees 2-theta±0.2 degrees 2-theta.

Crystalline Form C9 of Branaplam hydrochloride may alternatively be characterized by an XRPD pattern having peaks at 4.6, 9.1, 13.2, 13.8, 15.3, 15.7, 18.5, 22.4, and 23.6 and 26.6 degrees 2-theta±0.2 degrees 2-theta.

Crystalline Form C9 of Branaplam hydrochloride may be characterized by each of the above characteristics alone/or by all possible combinations, e.g., an XRPD pattern having peaks at 4.6, 13.2, 13.8, 18.5 and 23.6 degrees 2-theta±0.2 degrees 2-theta; an XRPD pattern as depicted in FIG. 9, and combinations thereof.

In another embodiment of the present disclosure, Branaplam hydrochloride Form C9 is polymorphically pure.

In an embodiment of the present disclosure, crystalline Form C9 of Branaplam hydrochloride is isolated.

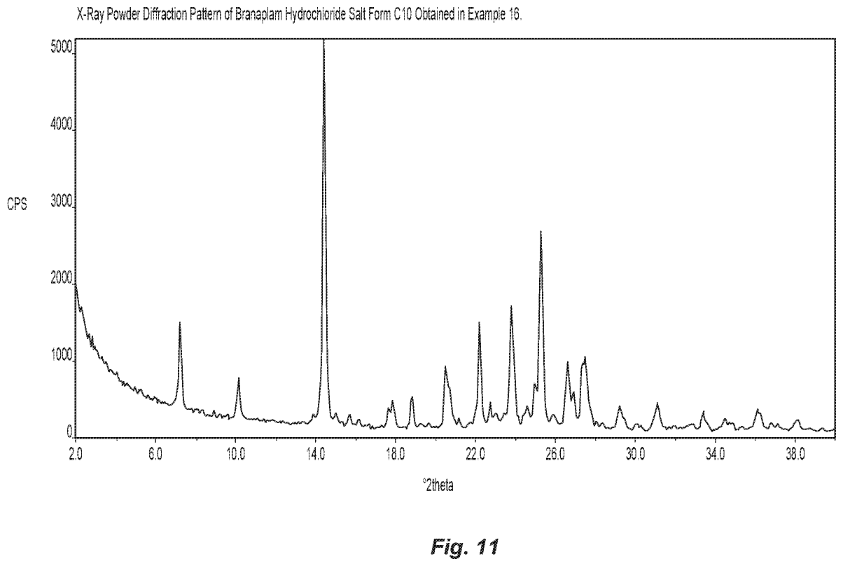

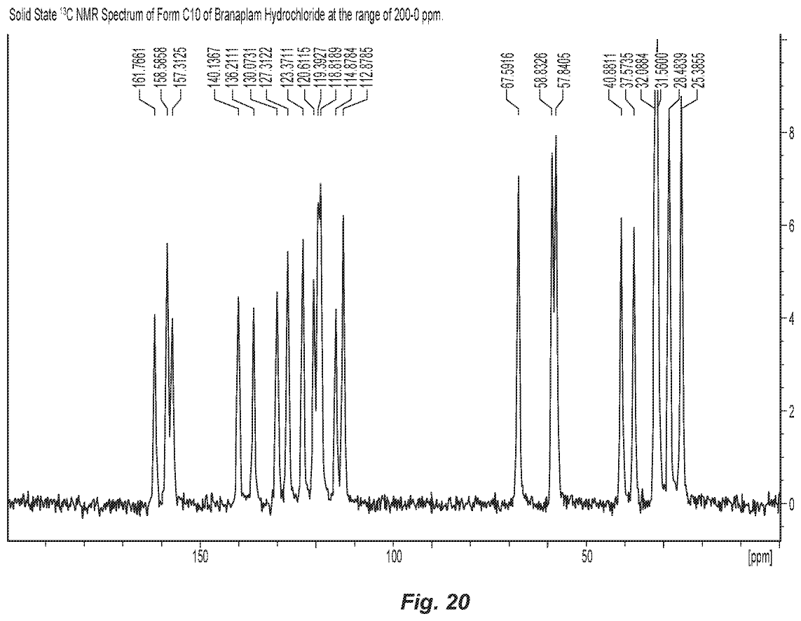

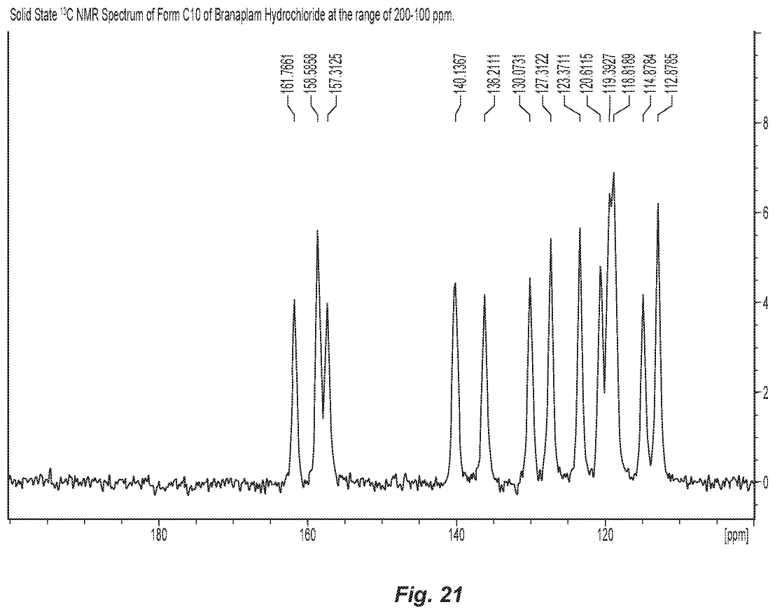

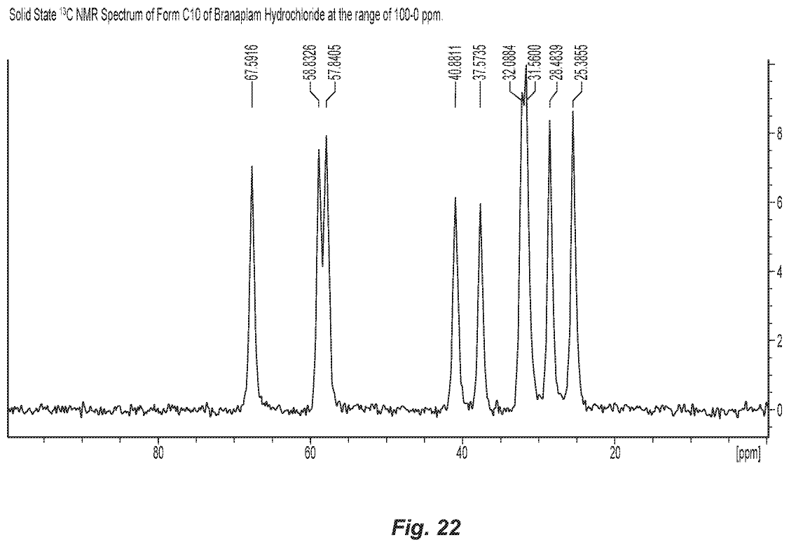

The present disclosure includes a crystalline polymorph of Branaplam hydrochloride, designated Form C10. The crystalline Form C10 of Branaplam hydrochloride may be characterized by data selected from one or more of the following: an X-ray powder diffraction pattern substantially as depicted in FIG. 11; an X-ray powder diffraction pattern having peaks at 7.3, 14.5, 22.3, 23.9 and 25.4 degrees 2-theta±0.2 degrees 2-theta; a solid state ¹³C-NMR spectrum having characteristic peaks at 140.2, 130.1, 120.6, 114.9 and 113.0 ppm±0.2 ppm; a solid state ¹³C-NMR spectrum having the following chemical shift absolute differences from a reference peak at 161.9 ppm±2 ppm of 21.7, 31.8, 41.3, 47.0 and 48.9 ppm±0.1 ppm; a solid state ¹³C-NMR spectrum as depicted in FIG. 20 or 21 or 22; and combinations of these data.

Crystalline Form C10 of Branaplam hydrochloride may be further characterized by an X-ray powder diffraction pattern having peaks at 7.3, 14.5, 22.3, 23.9 and 25.4 degrees 2-theta±0.2 degrees 2-theta, and also having any one, two, three, four or five additional peaks selected from 10.2, 17.9, 18.9, 20.6 and 26.7 degrees 2-theta±0.2 degrees 2-theta. In another embodiment Branaplam hydrochloride Form C10 can be characterized as anhydrous form.

Crystalline Form C10 of Branaplam hydrochloride may alternatively be characterized by an XRPD pattern having peaks at 7.3, 10.2, 14.5, 17.9, 18.9, 20.6, 22.3, 23.9, 25.4 and 26.7 degrees 2-theta±0.2 degrees 2-theta.

Crystalline Form C10 of Branaplam hydrochloride may be characterized by each of the above characteristics alone/or by all possible combinations, e.g., an XRPD pattern having peaks at 7.3, 14.5, 22.3, 23.9 and 25.4 degrees 2-theta±0.2 degrees 2-theta; an XRPD pattern as depicted in FIG. 11, and combinations thereof.

In another embodiment of the present disclosure, Branaplam hydrochloride Form C10 is polymorphically pure.

In an embodiment of the present disclosure, crystalline Form C10 of Branaplam hydrochloride is isolated.

As described above, depending on which other solid state it is compared with, Form C10 of Branaplam hydrochloride according to the present disclosure may have advantageous properties as described above. For example, Branaplam hydrochloride Form C10 is surprisingly stable and non-hygroscopic.

Branaplam hydrochloride Form C10 may be prepared by a process including crystallising Branaplam hydrochloride (such as amorphous Branaplam hydrochloride) in acetonitrile or in ethylacetate. The above Branaplam hydrochloride Form C10 can be prepared by a process including crystallising Branaplam hydrochloride (such as amorphous Branaplam hydrochloride) in acetonitrile. The process includes suspending Branaplam hydrochloride (such as amorphous Branaplam hydrochloride) in acetonitrile. In embodiments, about 30 to about 100 vol of acetonitrile, and in some embodiments about 40 to about 50 vol of acetonitrile is used for preparing the Branaplam hydrochloride suspension.

In embodiments, the suspension is heated to about 50 to about 83° C., in embodiments about 60 to about 83° C., and particularly about 83° C., optionally with stirring.

In embodiments, about 120 to about 190 vol of additional acetonitrile is added to the suspension, in embodiments about 120 to about 170 vol, and in some embodiments about 160 vol of acetonitrile is added in about 2 to about 6 portions, in an embodiment in about 4 portions. In embodiments, after the addition, the mixture is stirred for about 5 to about 30 minutes, in embodiments for about 15 minutes.

In embodiments, the suspension is cooled, in embodiments to room temperature before isolating the Branaplam hydrochloride Form C10. In embodiments, the suspension is maintained at room temperature for about 10 to about 60 hours, en embodiments for about 24 hours, optionally with stirring. In embodiments, prior to isolating the Branaplam hydrochloride Form C10, the suspension may be further cooled, in embodiments to about −30 to about 0° C., in embodiments about −20° C., optionally with stirring. The mixture may be held at this temperature for about 10 to about 100 hours, in embodiments about 72 hours, optionally with stirring.

In embodiments, the Branaplam hydrochloride Form C10 is isolated by filtration. The product may be optionally dried. Drying may be done by nitrogen or air or under vacuum. In embodiments, drying is performed at a temperature of about 30 to about 80° C., about 40 to about 60° C., about 40 to about 50° C., and particularly about 45° C. When the drying is under vacuum, a reduced pressure of: about 1 to about 200 mbar, about 1 to about 100 mbar, about 1 to about 50 mbar, particularly about 1 to about 20 mbar or about 10 mbar, is used.

Alternatively, the above Branaplam hydrochloride Form C10 can be prepared by a process including crystallising Branaplam hydrochloride (such as amorphous Branaplam hydrochloride) in ethyl acetate. In embodiments, the process includes suspending Branaplam hydrochloride (such as amorphous Branaplam hydrochloride) in ethyl acetate. In embodiments, about 10 to about 50 vol of ethyl acetate, in embodiments about 10 to about 40 vol, and in other embodiments about 40 vol of ethyl acetate is used for preparing Branaplam hydrochloride suspension.

Typically, the suspension is heated to about 50 to about 78° C., in embodiments about 65 to about 78° C., particularly about 75° C. The suspension may be maintained for an additional about 10 to about 100 hours, in embodiments for about 60 to about 90 hours and in other embodiments for about 80 hours, optionally with stirring.

In embodiments, prior to isolating the Branaplam hydrochloride Form C10, the suspension is cooled, in embodiments to room temperature and maintained at room temperature for about 10 to about 60 hours, particularly about 40 hours, optionally with stirring.

In embodiments, the Branaplam hydrochloride Form C10 may be isolated by filtration. The product may be optionally dried. Drying may be done by nitrogen or air or under vacuum. In embodiments, drying is performed at a temperature of about 30 to about 80° C. in embodiments about 40 to about 60° C., about 40 to about 50° C., and particularly about 45° C. When the drying is under vacuum, it may be at a reduced pressure of: about 1 to about 200 mbar, about 1 to about 100 mbar, about 1 to about 50 mbar, and particularly about 1 to about 20 mbar, and particularly about 10 mbar).

Alternatively, the preparation of Branaplam hydrochloride Form C10 may include crystallization of Branaplam hydrochloride in a mixture of DMAc (Dimethylacetamide) and toluene. The process includes:

a) combining a solution of Branaplam hydrochloride in DMAc (Dimethylacetamide), in embodiments with heating, with toluene;
b) optionally cooling the mixture; and
c) optionally isolating the Branaplam hydrochloride Form C10, and optionally drying.

Typically, about 30 to about 40 vol DMAc is used in stage a), in embodiments about 32 to about 37 vol and in embodiments about 35 vol. The mixture may be heated to a temperature of about 80 to about 120° C. and in embodiments about 110° C. The mixture may be stirred. The mixture may be filtered in order obtain a clear solution. After filtration, the mixture may be heated to a temperature of about 80 to about 100° C. and in embodiments about 90° C., prior to combining with toluene.

In embodiments, toluene is added to the solution of Branaplam hydrochloride in DMAc. In embodiments, the volume ratio of DMAc to toluene is about 1:1 to about 1:2, about 1:1 to about 1:1.5, in some embodiments 1:1 (v/v). In embodiments, the toluene is added in one-portion to the solution.

In embodiments, the clear mixture obtained in stage a) is cooled, in embodiments to a temperature of about 30 to about 50° C. and in some embodiments about 40° C. In embodiments the cooling is carried out over a period of about 1 to about 4 hours, about 1 to about 3 hours, and particularly about 2 hours. The solution may be maintained for about 1 to about 24 hours, about 2 to about 24 hours, and particularly about 3 hours, optionally under stirring.

In embodiments, the solution is further cooled to a temperature of about 2 to about 10° C., in embodiments about 4° C. An further portion of toluene may optionally be added to the cooled solution. In embodiments, toluene is added to provide a final volume ratio of DMAc to toluene of: about 1:1.1 to about 1:1.5, about 1:1.1 to about 1:1.3, in some embodiments about 1:1.1 (v/v). In embodiments, the toluene is added to the solution, and the solution is stirred at a temperature of about 2 to about 10° C., in embodiments about 4° C., in embodiments for about 1 to about 5 hours, in some embodiments about 3 to about 4 hours or particularly about 3.5 hours to obtain a suspension.

Isolation of Branaplam hydrochloride Form C10 may be done, for example, by filtering the suspension, and optionally drying. Drying may be done by nitrogen or air or under vacuum. Drying may be performed at a temperature of about 40 to about 60° C., in embodiments about 40 to about 50° C., and particularly about 45° C. When the drying is under vacuum, in embodiments reduced pressure of: about 1 to about 200 mbar, about 1 to about 100 mbar, about 1 to about 50 mbar, and particularly about 5 to about 40 mbar, and more particularly about 20 mbar, is used.

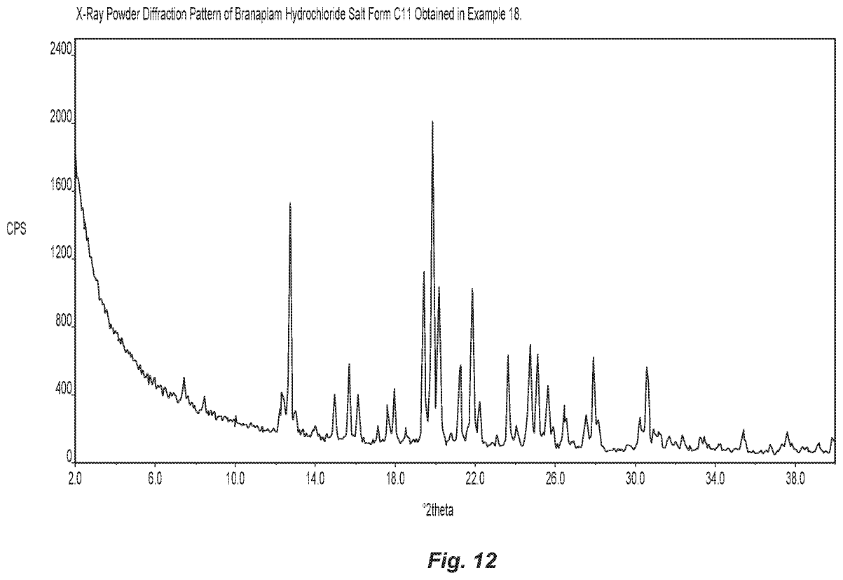

The present disclosure includes a crystalline polymorph of Branaplam hydrochloride, designated Form C11. The crystalline Form C11 of Branaplam hydrochloride may be characterized by data selected from one or more of the following: an X-ray powder diffraction pattern substantially as depicted in FIG. 12; an X-ray powder diffraction pattern having peaks at 12.8, 19.4, 19.9, 20.2 and 21.9 degrees 2-theta±0.2 degrees 2-theta; and combinations of these data.

Crystalline Form C11 of Branaplam hydrochloride may be further characterized by an X-ray powder diffraction pattern having peaks at 12.8, 19.4, 19.9, 20.2 and 21.9 degrees 2-theta±0.2 degrees 2-theta, and also having any one, two, three, four or five additional peaks selected from 15.0, 15.7, 16.2, 21.3 and 25.1 degrees 2-theta±0.2 degrees 2-theta.

Crystalline Form C11 of Branaplam hydrochloride may alternatively be characterized by an XRPD pattern having peaks at 12.8, 15.0, 15.7, 16.2, 19.4, 19.9, 20.2, 21.3, 21.9 and 25.1 degrees 2-theta±0.2 degrees 2-theta.

Crystalline Form C11 of Branaplam hydrochloride may be characterized by each of the above characteristics alone/or by all possible combinations, e.g., an XRPD pattern having peaks at 12.8, 19.4, 19.9, 20.2 and 21.9 degrees 2-theta±0.2 degrees 2-theta; an XRPD pattern as depicted in FIG. 12, and combinations thereof.

In another embodiment of the present disclosure, Branaplam hydrochloride Form C11 is polymorphically pure.

In an embodiment of the present disclosure, crystalline Form C11 of Branaplam hydrochloride is isolated.

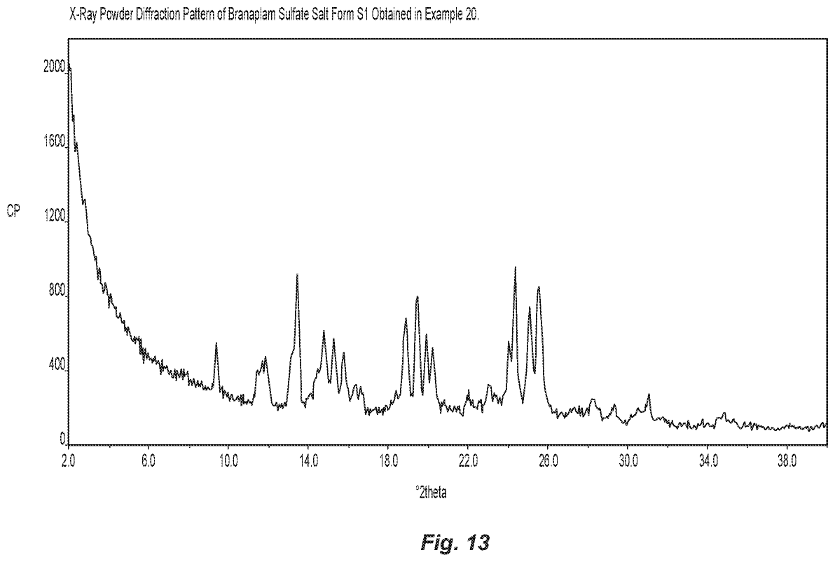

The present disclosure includes a crystalline polymorph of Branaplam Sulfate, designated Form S1. The crystalline Form S1 of Branaplam Sulfate may be characterized by data selected from one or more of the following: an X-ray powder diffraction pattern substantially as depicted in FIG. 13; an X-ray powder diffraction pattern having peaks at 9.4, 13.5, 18.8, 19.5 and 24.3 degrees 2-theta±0.2 degrees 2-theta; and combinations of these data.

Crystalline Form S1 of Branaplam Sulfate may be further characterized by an X-ray powder diffraction pattern having peaks at 9.4, 13.5, 18.8, 19.5 and 24.3 degrees 2-theta±0.2 degrees 2-theta, and also having any one, two, three, four or five additional peaks selected from 14.8, 15.3, 19.9, 20.2 and 25.0 degrees 2-theta±0.2 degrees 2-theta.

Crystalline Form S1 of Branaplam Sulfate may alternatively be characterized by an XRPD pattern having peaks at 9.4, 13.5, 14.8, 15.3, 18.8, 19.5, 19.9, 20.2, 24.3 and 25.0 degrees 2-theta±0.2 degrees 2-theta.

Crystalline Form S1 of Branaplam Sulfate may be characterized by each of the above characteristics alone/or by all possible combinations, e.g., an XRPD pattern having peaks at 9.4, 13.5, 18.8, 19.5 and 24.3 degrees 2-theta±0.2 degrees 2-theta; an XRPD pattern as depicted in FIG. 13, and combinations thereof.

The above Branaplam Sulfate Form S1 may be prepared by crystallising Branaplam sulfate in ethanol. In embodiments, the process includes suspending Branaplam sulfate in ethanol. The suspension of Branaplam sulfate in ethanol may be prepared by suspending Branaplam (such as Branaplam Form B1 as defined herein) in ethanol, and adding sulfuric acid to the suspension.

In embodiments, about 5 to about 30 vol of ethanol, in embodiments about 7 about 15 vol, and in some embodiments about 10 vol of ethanol is used for preparing Branaplam suspension. In embodiments the ethanol is absolute ethanol.

In embodiments, the suspension is kept at room temperature for about 5 to about 45 minutes, in embodiments about 10 to about 40 and in some embodiments about 30 minutes, optionally with stirring.

In embodiments, sulfuric acid (preferably 98%) is used in an amount of about 0.5 to about 1.2 molar equivalents sulfuric acid, in embodiments about 0.75 molar equivalents sulfuric acid is added to the suspension, optionally with stirring.

In embodiments, the suspension is kept at room temperature for about 5 to about 36 hours, in embodiments about 10 to about 24 hours and in some embodiments about 18 hours, optionally with stirring.

The Branaplam Sulfate Form S1 may be isolated. In embodiments isolation of Branaplam Sulfate Form S1 may be done, for example, by filtering the suspension, and optionally washing, in embodiments using ethanol. The Branaplam Sulfate Form S1 is optionally dried. Drying may be done by nitrogen or air or under vacuum. In embodiments, drying is performed at a temperature of about 20 to about 80° C., about 30 to about 60° C., about 40 to about 50° C., or about 45° C. When the drying is under vacuum, in embodiments a reduced pressure of: about 1 to about 200 mbar, about 1 to about 100 mbar, about 1 to about 50 mbar, particularly about 1 to about 20 mbar or about 10 mbar, is used.

In another embodiment of the present disclosure, Branaplam Sulfate Form S1 is polymorphically pure.

In an embodiment of the present disclosure, crystalline Form S1 of Branaplam Sulfate is isolated.

The above processes can further include a step of combining the Branaplam, Branaplam salts and crystalline forms thereof with at least one pharmaceutically acceptable excipient to prepare a pharmaceutical composition or a pharmaceutical formulation.

The present disclosure also relates to the uses of any one or a combination of the crystalline polymorphs of Branaplam and salts thereof, including hydrochloride and sulfate of the present disclosure, for preparing other crystalline polymorphs of Branaplam and salts thereof. For instance, Branaplam Form B1 can be used for the preparation of Branaplam Form B3 or Branaplam Form B5.

The present disclosure also relates to any one or a combination of the above described crystalline polymorphs of Branaplam and salts thereof, including hydrochloride and sulfate of the present disclosure, for use in the preparation of other crystalline polymorphs of Branaplam and salts thereof.

In another aspect, the present disclosure encompasses any one or combination of the above described crystalline polymorphs of Branaplam and salts thereof, including hydrochloride and sulfate, for use in the preparation of pharmaceutical compositions and/or formulations for use in medicine, in embodiments for the treatment of spinal muscular atrophy (SMA).

In another embodiment, the present disclosure also encompasses the uses of any one or combination of the above described crystalline polymorphs of Branaplam and salts thereof, including hydrochloride and sulfate, for the preparation of pharmaceutical compositions and/or formulations, preferably for use in medicine, preferably for the treatment of spinal muscular atrophy (SMA).

The present disclosure includes processes for preparing the above mentioned pharmaceutical compositions. The processes encompass combining any one of the above crystalline polymorphs of Branaplam and salts thereof and/or combinations thereof of the present disclosure with at least one pharmaceutically acceptable excipient.

Pharmaceutical formulations of the present invention contain any one or a combination of the crystalline polymorphs of Branaplam of the present invention, such as crystalline Branaplam Forms B1, B3, and/or B5 and crystalline Branaplam hydrochloride Forms C1, C3, C6 and C10. In addition to the active ingredient, the pharmaceutical formulations of the present invention can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present invention include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Branaplam and any other solid excipients are dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present invention, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present invention include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present invention is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present invention can be a capsule containing the composition, preferably a powdered or granulated solid composition of the invention, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, which causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, but they are not subjected to a final tableting step.

A pharmaceutical formulation of Branaplam can be administered. Branaplam is preferably formulated for administration to a mammal, preferably a human, by injection. Branaplam can be formulated, for example, as a viscous liquid solution or suspension, preferably a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Branaplam and the pharmaceutical compositions of Branaplam of the present disclosure can be used as medicaments, particularly in the treatment of spinal muscular atrophy (SMA).

In another embodiment, Branaplam Forms B1, B3 and B5 and/or Branaplam hydrochloride Forms C1, C3, C6 and C10, Branaplam sulfate Form S1 and/or the pharmaceutical compositions of Branaplam Forms B1, B3 and B5 and/or Branaplam hydrochloride Forms C1, C3, C6 and C10, and/or Branaplam sulfate Form S1 can be used as medicaments, such as in the treatment of spinal muscular atrophy (SMA).

The present disclosure also provides methods of treating spinal muscular atrophy (SMA) which include administering a therapeutically effective amount of crystalline polymorphs of Branaplam of the present disclosure, or at least one of the above pharmaceutical compositions, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Analytical Methods

### X-Ray Powder Diffraction (XRPD) Method

X-ray diffraction was performed on X-Ray powder diffractometer: Powder X-ray Diffraction was performed on ARL (SCINTAG) powder X-Ray diffractometer model X'TRA equipped with a solid state detector. Copper radiation of 1.5418 Å was used. Scanning parameters: range: 2-40 degrees two-theta; scan mode: continuous scan; step size: 0.05°, and a rate of 3 deg/min.

### Solid-State ¹³C NMR Method

Solid-state ¹³C NMR spectra were recorded with variable amplitude cross polarization, magic angle spinning and high power proton decoupling using a BRUKER Avance II+ spectrometer operating at 125 MHz and controlled temperature of 0° C. A probe using 4 mm o.d. zirconia rotors was employed. The operation conditions were: contact time 3 ms; acquisition time, recycle delay: 2 s, 5100 scans; spin rate: 11 kHz. Chemical shifts were referenced via a replacement sample of glycine (carboxyl carbon chemical shift assigned as 176.03 ppm relative to the signal of tetramethylsilane).

### EXAMPLES

Branaplam used herein can be prepared according to the procedure described in U.S. Pat. Nos. 8,729,263 and 9,545,404 Examples 17-13 which is describing Branaplam hydrochloride salt which can be converted to the corresponding Branaplam free base. CN patent application 103,965,169 is describing also procedure for preparation of Branaplam.

### Example 1. Preparation of Branaplam Form B1

Milli-Q water (ultra-pure water, 2 ml, 20V) was added to Branaplam (100 mg, 0.25 mmol). The obtained slurry was kept at 90° C. for about 18 hours under stirring. Next, the slurry was cooled to room temperature, filtered under vacuum and dried in vacuum oven at 45° C. for 18 hours to afford off-white solid, which was confirmed to contain Branaplam Form B1 according to X-ray powder diffraction as shown in FIG. 1.

### Example 2. Preparation of Branaplam Form B3

DMAc (N,N-dimethylacetamide) (0.3 ml, 20V) was added to Branaplam (15 mg, 0.038 mmol) to obtain slurry. The slurry was heated to 110° C. over a period of 30 minutes to obtain turbid solution followed by hot filtration at about 110° C. to obtain clear filtrate solution. The clear filtrate solution was cooled to room temperature and stirred at room temperature for 18 hours to give a precipitation. The precipitation was then filtered to afford off-white solid. The obtained product was characterized by X-ray powder diffraction as Branaplam Form B3.

### Example 3. Preparation of Branaplam Form B3

Branaplam Form B1 (about 5 mg) were placed into T zero Aluminum Pan. The sample was heated at rate of 10° C./min until 250° C. under nitrogen atmosphere (about 50 ml/min N2 flow) using DSC instrument. The sample was characterized by X-ray powder diffraction to afford Branaplam Form B3.

### Example 4. Preparation of Branaplam Form B1

Double distilled water (0.4 ml, 20V) was added to Branaplam hydrochloride salt Form C1 (20 mg, 0.0465 mmol) to obtain slurry. The obtained slurry was magnetically stirred at 80° C. for 5 minutes. Next, the hot slurry was alkalized with 1N NaOH (0.04 ml) to give pH-9 and continued for additional 5 minutes with stirring at 80° C. Then, the reaction mixture was cooled to room temperature and filtrated under centrifuge to give off-white solid, which was characterized by X-ray powder diffraction as Branaplam Form B 1.

### Example 5. Preparation of Branaplam Form B1

2,2,2-Trifluoroethanol (1.25 ml, 25V) was added to Branaplam (50 mg, 0.127 mmol) and heated to 75° C. over a period of 30 minutes with stirring to obtain complete dissolution follows by mechanically filtration using filter disk. Next, the clear filtrate and water as antisolvent (1.25 ml) were added drop-wise simultaneously at the same rate into a vessel under mixing at room temperature. A precipitation was observed immediately during the simultaneous addition at room temperature. The obtained slurry was stirred at room temperature over a period of 18 hours. The precipitation was then filtered by centrifuge to afford off-white solid, which was characterized by X-ray powder diffraction as Branaplam Form B 1.

### Example 6. Preparation Branaplam Form B5

2,2,2-Trifluoroethanol (5.3 ml, 106V) was added to Branaplam (50 mg, 0.13 mmol) to give complete dissolution at room temperature. The clear solution was mechanically filtrated by filter disk. The obtained clear solution was slowly evaporated at room temperature over a period of 5 days. The obtained solid was dried in a vacuum oven at 45° C. for 5 hours to afford off-white to gray solid, which was characterized by X-ray powder diffraction as Branaplam Form B5 (FIG. 3).

### Example 7. Preparation of Branaplam Hydrochloride Form C1

A round flask was loaded with absolute ethanol (6 ml, 10V) and Branaplam form B1 was added (600 mg, 1.52 mmol) to obtain slurry. The obtained slurry was magnetically stirred at room temperature and a solution of 1.25M HCl in ethanol (1.83 ml, 2.28 mmol) was added immediately in one portion to the stirred slurry. The slurry was stirred at room temperature over a period of 15 hours followed by additional stirring at 0° C. for 30 minutes. Next, the slurry was filtrated under vacuum and washed twice with ethanol (21.2 ml). To the obtained solid, ethanol (6 ml, 10 V) was added to give slurry that was stirred at room temperature over a period of 3 hours, filtered under vacuum and washed twice with ethanol (2*1.2 ml). The obtained solid was dried in a vacuum oven at 45° C. for 16 hours to afford off-white to gray solid, which was characterized by X-ray powder diffraction as Branaplam hydrochloride Form C1.

### Example 8. Preparation of Branaplam Hydrochloride Form C1

A round flask was loaded with absolute ethanol (30 ml, 10V) and Branaplam Form B3 (3 g, 7.62 mmol) to give slurry. The obtained slurry was magnetically stirred at room temperature and a solution of 1.25M HCl in ethanol (9.15 ml, 11.43 mmol) was immediately added in one portion to the stirred slurry followed by addition of absolute ethanol (8 ml, 2.7V). The slurry was stirred at room temperature over a period of 18 hours followed by stirring at 0° C. for 30 minutes. Next, the slurry was filtrated under vacuum and washed twice with ethanol (2*3 ml). To the obtained solid, ethanol (30 ml, 10 V) was added to give slurry at room temperature. The slurry was stirred at room temperature over a period of 4.5 hours, filtered under vacuum and washed twice with ethanol (2*6 ml). The obtained solid was dried in a vacuum oven at 45° C. for 16 hours to afford off-white to gray solid, which was characterized by X-ray powder diffraction as Branaplam hydrochloride Form C1 (FIG. 4).

### Example 9. Preparation of Branaplam Hydrochloride Form C3

Acetonitrile (20 ml, 400V) was added to Branaplam hydrochloride Form C1 (50 mg, 0.116 mmol) at room temperature to give slurry. The obtained slurry was magnetically stirred at room temperature for about 5 minutes followed by stirring at 60° C. for additional 5 minutes. Then, the hot slurry was filtrated under vacuum. The obtained solid was dried in a vacuum oven at 45° C. over a period of 18 hours to afford off-white solid, which was characterized by X-ray powder diffraction as Branaplam hydrochloride Form C3 (FIG. 5).

### Example 10. Preparation of Branaplam Hydrochloride Form C2

Acetonitrile (20 ml, 400V) was added to Branaplam hydrochloride Form C1 (50 mg, 0.116 mmol) to give diluted slurry. The obtained diluted slurry was magnetically stirred at room temperature for about 5 minutes. Next, the slurry was cooled to 4° C. and continued to stir upon cooling for additional ˜3 hours. Then, the cold slurry was filtered by centrifuge to give a solid, which was characterized by X-ray powder diffraction as Branaplam hydrochloride Form C2.

### Example 11. Preparation of Branaplam Hydrochloride Form C6

1-propanol (20 ml, 400V) was added to Branaplam hydrochloride Form C1 (50 mg, 0.116 mmol) to give slurry. The obtained slurry was magnetically stirred at room temperature for about 5 minutes. Next, the slurry was heated to 60° C. and continued to stir upon heating for additional ˜10 minutes. Then, the hot slurry was filtered under vacuum to give a solid, which was characterized by X-ray powder diffraction as Branaplam hydrochloride Form C6. This procedure for the preparation of form C6 can also use different solvents, such as: 2-isopropanol, 2-butanol, cellosolve and/or ethanol, instead of 1-propanol, using the same detailed procedure above.

### Example 12. Preparation of Branaplam Hydrochloride Form C8

Methanol (600 ml, 200V) was added to Branaplam hydrochloride Form C6 (3 g, 6.98 mmol) to give diluted slurry. The obtained diluted slurry was magnetically stirred at room temperature for about 15 minutes to give clear solution. Next, the solution was mechanically filtered under vacuum. The obtained clear mother-liquor was evaporated upon 60° C. and 300-350 mbar to give a solid, which was characterized by X-ray powder diffraction as Branaplam hydrochloride Form C8.

### Example 13. Preparation of Branaplam Hydrochloride Form C9

Water (1.5 ml, 15V) was added to Branaplam hydrochloride Form C8 (0.1 g, 0.23 mmol) to give slurry. The obtained slurry was magnetically stirred at room temperature for 40 minutes. Next, the slurry was filtered by centrifuge to isolate a solid, which was characterized by X-ray powder diffraction as Branaplam hydrochloride Form C9.

### Example 14. Preparation of Branaplam Hydrochloride Form C9

Branaplam hydrochloride Form C8 (about 200 mg) was grinded using mortar and pestle with 2 drops of water for 1 minute at room temperature. The obtained solid was characterized by X-ray powder diffraction as Branaplam hydrochloride Form C9.

### Example 15. Preparation of Branaplam Hydrochloride Form C9 Using Amorphous Branaplam Hydrochloride

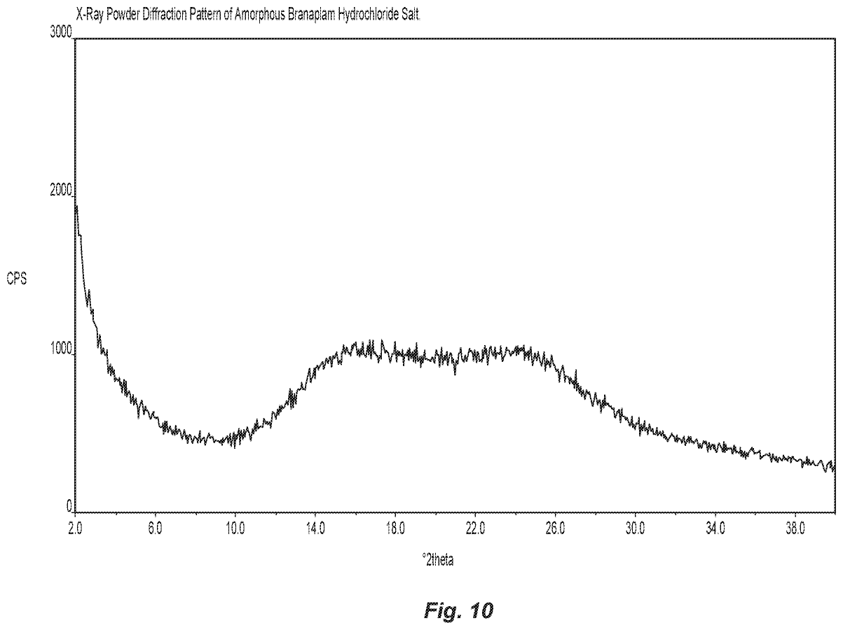

Methanol (60 ml, 60V) and dichloromethane (20 ml, 20V) were added to Branaplam hydrochloride Form C6 (1 g, 2.33 mmol) to give diluted slurry. The obtained diluted slurry was magnetically stirred at room temperature for 15 minutes to give clear solution. Then, the solution was filtered under vacuum and the clear mother-liquor was dried by spray dryer to give amorphous Branaplam hydrochloride. XRD measurement confirmed amorphous Branaplam hydrochloride content (FIG. 10). In the next step, the amorphous Branaplam hydrochloride (about 200 mg) was grinded with 2 drops of water for 1 minute at room temperature using mortar and pestle. The obtained solid was characterized by X-ray powder diffraction as Branaplam hydrochloride Form C9.

### Example 16. Preparation of Branaplam Hydrochloride Form C10

Acetonitrile (4 ml, 40V) was added to amorphous Branaplam hydrochloride (100 mg, 0.233 mmol, prepared according to procedure in example 15) to give slurry. The obtained slurry was magnetically stirred at room temperature over a period of 20 minutes and then heated to 83° C. Then, four portions of acetonitrile (4*4 ml, 4*40V) were added gradually to the stirred slurry in a rate of 40V per 5 minutes while stirring upon 83° C. and the slurry continued to stir at this temperature for additional 15 minutes. Then, the slurry was spontaneously cooled to room temperature. The slurry stirred at room temperature over a period of 24 hours follows by cooling to −20° C. for 72 hours. The cold slurry was filtered by centrifuge to give a solid, which was dried in vacuum oven at 45° C. for ˜18 hours and characterized by X-ray powder diffraction as Branaplam hydrochloride Form C10 (FIG. 11).

### Example 17. Preparation of Branaplam Hydrochloride Form C10

Ethyl acetate (1.5 ml, 30V) was added to amorphous Branaplam hydrochloride (50 mg, 0.116 mmol, can be prepared according to Example 15) to give slurry. The obtained slurry was mechanically stirred at a rate of 700 rpm in crystalline at room temperature for about 10-15 minutes. Then, the slurry was heated to 75° C. and stirred upon heating over a period of 80 hours. Subsequently, the slurry was spontaneously cooled to 25° C. without stirring. The slurry remained in this condition without stirring and at room temperature for 40 hours follows by filtration. The isolated solid was dried in vacuum oven at 45° C. for 24 hours and characterized by X-ray powder diffraction as Branaplam hydrochloride Form C10.

### Example 18. Preparation of Branaplam Hydrochloride Form C11

Dimethyl sulfoxide (0.350 ml, 7V) was added to anhydrous Branaplam hydrochloride (50 mg, 0.116 mmol, for example form C3 and C10, or mixtures thereof) to obtain slurry. The slurry was magnetically stirred and heated to 110° C. over a period of 15 minutes to give clear yellow solution. The obtained hot clear solution was mechanically filtrated upon filter disk. The obtained clear filtrate was then heated again to about 100° C. follows by addition of heptane (0.5 ml, 10V, in one portion) to the hot clear solution to give bi-phases solution. The obtained bi-phases solution was spontaneously cooled to 40° C.; at this temperature turbidity was observed. The turbid solution was stirred at 40° C. over a period of ˜48 hours and the obtained precipitation was then filtered by centrifuge follows by drying in vacuum oven at 45° C. for ˜16 hours to afford solid, which was characterized by X-ray powder diffraction as Branaplam hydrochloride Form C11.

### Example 19. Preparation of Branaplam Hydrochloride Form C11

Dimethyl sulfoxide (1.55 ml, 7V) was added to anhydrous Branaplam hydrochloride (222 mg, 0.52 mmol, for example form C3 and form C10 or mixtures thereof) to obtain slurry. The slurry was magnetically stirred and heated to 110° C. over a period of −15 minutes to give clear yellow solution. The obtained hot clear solution was mechanically filtrated upon filter disk. The obtained clear filtrate was then heated again to −90° C. follows by addition of toluene (2.22 ml, 10V) in one portion to the hot clear solution to give spontaneously precipitation. The obtained slurry was cooled to 40° C. over a period of −23 hours follows by cooling to 25° C. Then, the slurry was filtered by centrifuge follows by drying in vacuum oven at 45° C. during ˜20 hours to afford solid, which was characterized by X-ray powder diffraction as Branaplam hydrochloride Form C 11.

### Example 20. Preparation of Branaplam Sulfate Form S1

A round flask was loaded with absolute ethanol (3 ml, 10V) and Branaplam form B1 (300 mg, 0.76 mmol) to obtain slurry. The obtained slurry was magnetically stirred at room temperature for 0.5 h and then 98% H2SO4 (30 μl, 0.57 mmol, 0.75 eq) was added in one portion to the stirred slurry. The slurry was stirred at room temperature for 18 hours. Next, the slurry was filtrated under vacuum and washed three times with ethanol (3*3 ml). The obtained solid was dried in vacuum oven at 45° C. for 72 hours to afford yellow solid, which was characterized by X-ray powder diffraction as Branaplam sulfate Form S1.

### Example 21. Preparation of Branaplam Form B3

DMSO (Dimethyl Sulfoxide) (8 ml, 20V) was added to Branaplam (400 mg, 1.02 mmol) to give slurry at room temperature. The slurry was magnetically stirred and heated to about 120° C. over a period of 20 minutes to give complete dissolution followed by hot mechanically filtration through filter disk. Then, methyl acetate (4 ml, 10V) was added in one portion to the clear light brown filtrate. The clear solution was cooled spontaneously to room temperature and stirred at room temperature for about 16 hours to give a precipitant. The precipitation was then filtered to afford solid, which was dried in vacuum oven at 45° C. for about 18 hours. The dried solid was characterized by X-ray powder diffraction as Branaplam Form B3.

### Example 22. Preparation of Branaplam Hydrochloride Form C1

Water (40 ml, 20V) was added to Branaplam hydrochloride Form C6 (2 grams, 4.65 mmol) to give slurry at room temperature. The slurry was magnetically stirred at room temperature over a period of about 8.5 hours. Then, the slurry was vacuum filtrated to afford solid, which was dried in vacuum oven at 45° C. for about 14 hours. The dried solid was characterized by X-ray powder diffraction as Branaplam Form C1.

### Example 23. Preparation of Branaplam Hydrochloride Form C1

DMSO (Dimethyl Sulfoxide) (2.31 ml, 7V) was added to Branaplam hydrochloride (330 mg, 0.77 mmol) to give slurry at room temperature. The slurry was magnetically stirred and heated to about 110° C. over a period of 30 minutes to give complete dissolution followed by hot mechanically filtration through filter disk. Then, water (3.33 ml, 10V) was added drop-wise to the clear light brown filtrate. The clear solution was cooled spontaneously to room temperature and stirred at room temperature for about 11 hours to give a precipitant. The precipitation was then vacuum filtered to afford solid, which was dried in vacuum oven at 45° C. for about 12.5 hours. The dried solid was characterized by X-ray powder diffraction as Branaplam Form C1.

### Example 24. Preparation of Branaplam Hydrochloride Form C3

Ethyl acetate (18 ml, 30V) was added to Branaplam hydrochloride Form C1 (0.6 grams, 1.4 mmol) to give slurry at room temperature. The slurry was heated and magnetically stirred at about 75° C. for 24 hours. Then, the slurry was cooled spontaneously to room temperature followed by vacuum filtration to afford solid, which was dried in vacuum oven at 45° C. for about 15 hours. The dried solid was characterized by X-ray powder diffraction as Branaplam Form C3.

### Example 25. Preparation of Branaplam Hydrochloride Form C10

DMAc (Dimethylacetamide) (1.05 ml, 35V) was added to Branaplam hydrochloride (0.03 grams, 0.07 mmol) to give slurry at room temperature. The slurry was heated and magnetically stirred at about 110° C. to give clear solution followed by hot mechanically filtration through filter disk. Then, the clear filtrate solution was stirred and heated to about 90° C. followed by one-portion addition of toluene (1.05 ml, 35V). The clear mixture was cooled to 40° C. during about 2 hours and maintained upon stirring at this temperature over a period of 3 hours. Then, the clear mixture was cooled to 4° C. and additional portion of toluene (0.15 ml, 5V) was added followed by stirring at 4° C. during about 3.5 hours to give a precipitant. The precipitation was then filtered by centrifuge to afford solid, which was dried in vacuum oven at 45° C. for about 20 hours. The dried solid was characterized by X-ray powder diffraction as Branaplam Form C10.

1. A crystalline form of Branaplam hydrochloride designated as Form C1, characterized by data selected from one or more of the following:
a. an X-ray powder diffraction pattern having peaks at 4.5, 15.0, 16.6, 17.7 and 25.9 degrees 2-theta±0.2 degrees 2-theta;
b. an XRPD pattern as depicted in FIG. 4;
c. a solid state ¹³C-NMR spectrum having characteristic peaks at 160.0, 134.1, 126.5, 118.4 and 113.6 ppm±0.2 ppm;
d. a solid state ¹³C-NMR spectrum having the following chemical shift absolute differences from a reference peak at 162.1 ppm±2 ppm of 2.1, 28.0, 35.6, 43.7 and 48.5 ppm±0.1 ppm;
e. a solid state ¹³C-NMR spectrum as depicted in FIG. 14 or 15 or 16; and
f. a combination of any two or more of the above.

2. The crystalline Form C1 of Branaplam hydrochloride according to claim 1, characterized by an XRPD pattern having peaks at 4.5, 15.0, 16.6, 17.7 and 25.9 degrees 2-theta±0.2 degrees 2-theta, and also having any one, two, three, four or five additional peaks selected from 11.3, 13.5, 14.3, 24.0 and 28.5 degrees 2-theta±0.2 degrees 2-theta.

3. A crystalline form of Branaplam hydrochloride designated as Form C3, characterized by data selected from one or more of the following:
a. an X-ray powder diffraction pattern having peaks at 5.6, 13.7, 15.6, 18.3 and 24.7 degrees 2-theta±0.2 degrees 2-theta;
b. an XRPD pattern as depicted in FIG. 5;
c. a solid state ¹³C-NMR spectrum having characteristic peaks at 156.4, 135.6, 124.8, 121.8 and 120.2 ppm±0.2 ppm;
d. a solid state ¹³C-NMR spectrum having the following chemical shift absolute differences from a reference peak at 163.0 ppm±2 ppm of 6.6, 27.4, 38.2, 41.2 and 42.8 ppm±0.1 ppm;
e. a solid state ¹³C-NMR spectrum as depicted in FIG. 17 or 18 or 19; and
f. a combination of any two or more of the above.

4. The crystalline Form C3 of Branaplam hydrochloride according to claim 3, characterized by an XRPD pattern having peaks at 5.6, 13.7, 15.6, 18.3 and 24.7 degrees 2-theta±0.2 degrees 2-theta, and also having any one, two, three, four or five additional peaks selected from 12.6, 14.7, 20.0, 22.1 and 23.8 degrees two theta±0.2 degrees two-theta.

5. A crystalline form of Branaplam hydrochloride designated as Form C10, characterized by data selected from one or more of the following:
a. an X-ray powder diffraction pattern having peaks at 7.3, 14.5, 22.3, 23.9 and 25.4 degrees 2-theta±0.2 degrees 2-theta;
b. an XRPD pattern as depicted in FIG. 11;
c. a solid state ¹³C-NMR spectrum having characteristic peaks at 140.2, 130.1, 120.6, 114.9 and 113.0 ppm±0.2 ppm;
d. a solid state ¹³C-NMR spectrum having the following chemical shift absolute differences from a reference peak at 161.9 ppm±2 ppm of 21.7, 31.8, 41.3, 47.0 and 48.9 ppm±0.1 ppm;
e. a solid state ¹³C-NMR spectrum as depicted in FIG. 20 or 21 or 22; and
f. a combination of any two or more of the above.

6. The crystalline Form C10 of Branaplam hydrochloride according to claim 5, characterized by an XRPD pattern having peaks at 7.3, 14.5, 22.3, 23.9 and 25.4 degrees 2-theta±0.2 degrees 2-theta, and also having any one, two, three, four or five additional peaks selected from 10.2, 17.9, 18.9, 20.6 and 26.7 degrees 2-theta±0.2 degrees 2-theta.

7. A crystalline form of Branaplam designated as Form B 1, characterized by data selected from one or more of the following:
a. an X-ray powder diffraction pattern having peaks at 3.7, 7.5, 16.4, 24.8 and 26.0 degrees 2-theta±0.2 degrees 2-theta;
b. an XRPD pattern as depicted in FIG. 1;
c. a combination of the above.

8. The crystalline Form B1 of Branaplam according to claim 7, characterized by an XRPD pattern having peaks at 3.7, 7.5, 16.4, 24.8 and 26.0 degrees 2-theta±0.2 degrees 2-theta, and also having any one, two, three, four or five additional peaks selected from 13.8, 15.0, 18.2, 20.1 and 20.7 degrees two theta±0.2 degrees two theta.

9. A crystalline form of Branaplam designated as Form B3, characterized by data selected from one or more of the following:
a. an X-ray powder diffraction pattern having peaks at 11.5, 12.9, 15.5, 19.0 and 28.8 degrees two theta±0.2 degrees two-theta;
b. an XRPD pattern as depicted in FIG. 2;
c. a combination of the above.

10. The crystalline Form B3 of Branaplam according to claim 9, characterized by an XRPD pattern having peaks at 11.5, 12.9, 15.5, 19.0 and 28.8 degrees 2-theta±0.2 degrees 2-theta, and also having any one, two, three, four or five additional peaks selected from 9.3, 14.6, 17.5, 19.8 and 21.7 degrees two theta±0.2 degrees two-theta.

11. A crystalline form of Branaplam designated as Form B5, characterized by data selected from one or more of the following:
a. an X-ray powder diffraction pattern having peaks at 6.4, 9.5, 15.9, 19.0 and 23.5 degrees 2-theta±0.2 degrees 2-theta;
b. an XRPD pattern as depicted in FIG. 3;
c. a combination of the above.

12. The crystalline Form B5 of Branaplam according to claim 11, characterized by an XRPD pattern having peaks at 6.4, 9.5, 15.9, 19.0 and 23.5 degrees 2-theta±0.2 degrees 2-theta, and also having any one, two, three, four or five additional peaks selected from 3.2, 14.2, 20.9, 25.5 and 26.4 degrees 2-theta±0.2 degrees 2-theta.

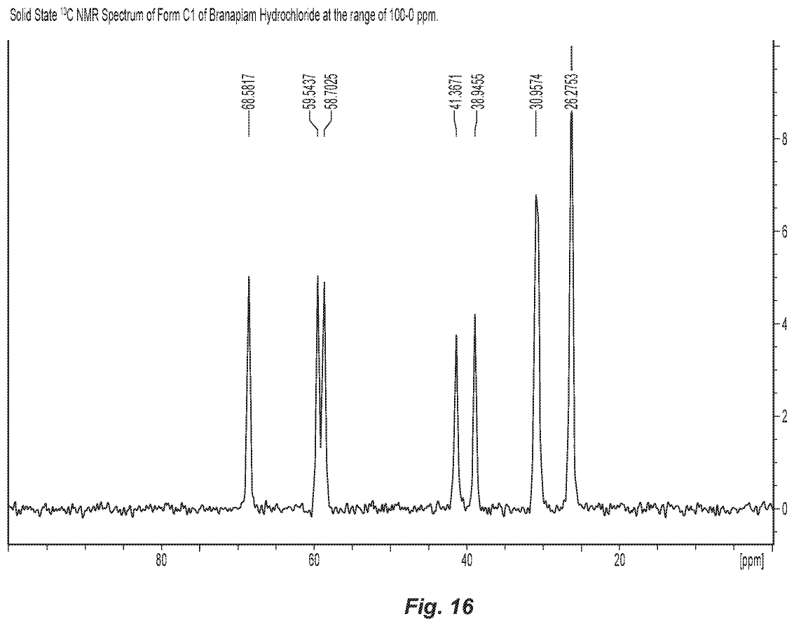

13. A crystalline form of Branaplam sulfate designated as Form S1, characterized by data selected from one or more of the following:
a. an X-ray powder diffraction pattern having peaks at 9.4, 13.5, 18.8, 19.5 and 24.3 degrees 2-theta±0.2 degrees 2-theta;
b. an XRPD pattern as depicted in FIG. 16;
c. a combination of the above.

14. The crystalline Form S1 of Branaplam sulfate according to claim 13, characterized by an XRPD pattern having peaks at 9.4, 13.5, 18.8, 19.5 and 24.3 degrees 2-theta±0.2 degrees 2-theta, and also having any one, two, three, four or five additional peaks selected from 14.8, 15.3, 19.9, 20.2 and 25.0 degrees 2-theta±0.2 degrees 2-theta.

15. A crystalline form of Branaplam hydrochloride designated as Form C6, characterized by data selected from one or more of the following:
a. an X-ray powder diffraction pattern having peaks at 6.4, 16.9, 19.4, 24.2 and 25.8 degrees 2-theta±0.2 degrees 2-theta;
b. an XRPD pattern as depicted in FIG. 6;
c. a combination of the above.

16. The crystalline Form C6 of Branaplam hydrochloride according to claim 15, characterized by an XRPD pattern having peaks at 6.4, 16.9, 19.4, 24.2 and 25.8 degrees 2-theta±0.2 degrees 2-theta, and also having any one, two, three, four or five additional peaks selected from 8.2, 9.8, 10.6, 15.3, and 22.7 degrees 2-theta±0.2 degrees 2-theta.

17. A pharmaceutical composition comprising a crystalline form according to claim 1.

18. (canceled)

19. A pharmaceutical formulation comprising a crystalline form according to claim 1, and at least one pharmaceutically acceptable excipient.

20. A process for preparing the pharmaceutical formulation according to claim 19, comprising combining the crystalline form with at least one pharmaceutically acceptable excipient.

21. A medicament comprising the crystalline form according to claim 1.

22. (canceled)

23. A method of treating spinal muscular atrophy (SMA), comprising administering a therapeutically effective amount of a crystalline form according to claim 1, to a subject suffering from spinal muscular atrophy (SMA), or otherwise in need of the treatment.

24. (canceled)

25. A process for preparing Branaplam hydrochloride Form C1 as defined in claim 1, comprising crystallising Branaplam hydrochloride in a mixture of DMSO (dimethyl sulfoxide) and water.

26. A process according to claim 25 comprising:
a) combining a solution of Branaplam hydrochloride in DMSO optionally with heating, with water;
b) optionally cooling; and
c) optionally isolating the Branaplam hydrochloride Form C1.

27. A process according to claim 26, wherein the DMSO is present in the reaction mixture an amount of about 5 to about 12 vol relative to Branaplam.

28. A process according to any of claim 26, wherein the Branaplam solution in DMSO is at a temperature of about 80° C. to about 120° C.

29. A process according to claim 26 wherein water is added to the solution of Branaplam in DMSO at a volume ratio of DMSO to water from about 1:1 to about 1:2 (v/v).

30. A process according to claim 26, wherein the reaction mixture is cooled to room temperature and maintained for about 5 to 24 hours, optionally with stirring.

31. A process according to claim 26, wherein the Branaplam hydrochloride Form C1 is isolated, and optionally washed, and optionally dried.

32. A process according to claim 31, wherein the drying is carried out under reduced pressure at a temperature of about 40 to about 60° C.

33. A process according to claim 25, further comprising a step of combining the Branaplam hydrochloride Form C1 with at least one pharmaceutically acceptable excipient to form a pharmaceutical composition or a pharmaceutical formulation.

34. A process for preparing Branaplam hydrochloride Form C3 as defined in claim 3, comprising suspending Branaplam hydrochloride Form C1 in ethyl acetate, and optionally isolating the Branaplam hydrochloride Form C3.

35. A process according to claim 34, wherein the ethyl acetate is present in the reaction mixture in an amount of about 15 to about 45 vol relative to the Branaplam hydrochloride.

36. A process according to claim 34, wherein the Branaplam suspension is heated to a temperature of about 50° C. to about 90° C.

37. A process according to claim 34, wherein the Branaplam suspension is maintained at a temperature of about 50° C. to about 90° C., for about 10 to 60 hours, optionally with stirring.

38. A process according to claim 34, wherein the slurry is cooled to room temperature.

39. A process according to claim 34, wherein the Branaplam hydrochloride form C3 is isolated, optionally washed, and optionally dried.

40. A process according to claim 39, wherein the drying is carried out under reduced pressure at a temperature of about 30 to about 80° C.

41. A process according to claim 34, further comprising a step of combining Branaplam hydrochloride Form C3 with at least one pharmaceutically acceptable excipient to form a pharmaceutical composition or a pharmaceutical formulation.

42. A process for preparing Branaplam hydrochloride Form C10 as defined in claim 5, comprising crystallizing Branaplam hydrochloride in a mixture of DMAc (Dimethylacetamide) and toluene.

43. A process according to claim 42, comprising:
a) combining a solution of Branaplam hydrochloride in DMAc, with heating, with toluene;
b) optionally cooling the mixture; and
c) optionally isolating the Branaplam hydrochloride Form C10, and optionally drying.

44. A process according to claim 42, wherein about 30 to about 40 vol DMAc is used in stage a).

45. A process according to claim 42, wherein the solution of Branaplam hydrochloride in DMAc in step (a) is heated to a temperature of about 80 to about 120° C. optionally with stirring.

46. A process according to claim 45, wherein the mixture is filtered.

47. A process according claim 46, wherein the mixture is heated to a temperature of about 80 to about 100° C., prior to combining with toluene.

48. A process according to claim 43, wherein toluene is added to the solution of Branaplam hydrochloride in DMAc so the volume ratio of DMAc to toluene is about 1:1 to about 1:2 (v/v).

49. A process according to claim 43, wherein the mixture obtained in step a) is cooled to a temperature of about 30-50° C. over a period of about 1 to about 4 hours.

50. A process according to claim 43, wherein the mixture is maintained for about 1 to about 24 hours, optionally with stirring.

51. A process according to claim 49, wherein the solution is further cooled to a temperature of about 2 to about 10° C.

52. A process according to claim 51, wherein a further portion of toluene is added to the cooled solution to provide a final volume ratio of DMAc to toluene of about 1:1.1 to about 1:1.5 (v/v).

53. A process according to claim 52, wherein the toluene is added to the solution, and the solution is stirred at a temperature of about 2 to about 10° C. for about 1 to about 5 hours to obtain a suspension.

54. A process according to claim 43, wherein the isolation of Branaplam hydrochloride Form C10 comprises filtering the suspension, and optionally drying.

55. A process according to claim 43, wherein the drying is carried out under reduced pressure at a temperature of about 40 to about 60° C.

56. A process according to claim 42, further comprising a step of combining Branaplam hydrochloride Form C10 with at least one pharmaceutically acceptable excipient to form a pharmaceutical composition or a pharmaceutical formulation.
